(19) United States
(12) Patent Application Publication
(10) Pub. No.: US 2011/0021541 A1
(43) Pub. Date: Jan. 27, 2011

(54) INHIBITORS OF HUMAN PHOSPHATIDYL-INOSITOL 3-KINASE DELTA

(76) Inventors: Stephen L. White, Boyds, MD (US); Fuqiang Ruan, Bellevue, WA (US); Edward A. Kesicki, Bothell, WA (US); Eugene Thorsett, Half Moon Bay, CA (US); Francine Farouz, Mercer Island, WA (US)

Correspondence Address: MORRISON & FOERSTER LLP, 12531 HIGH BLUFF DRIVE, SUITE 100, SAN DIEGO, CA, 92130-2040, US

(21) Appl. No.: 12/742,904

(22) PCT Filed: Nov. 12, 2008
(86) PCT No.: PCT/US08/83262

§ 371 Date: Sep. 23, 2010

Related U.S. Application Data
(60) Provisional application No. 60/987,723, filed on Nov. 13, 2007.

(51) Int. Cl. A61K 31/52; C07D 473/34; C12N 5/078; C12N 9/99; C07D 473/38; A61P 19/08; A61P 35/00; A61P 11/06; A61P 19/02; A61P 25/00; A61P 9/12; A61P 35/02

(52) U.S. Cl. 514/260.1; 544/277; 435/375; 435/184; 514/263.21; 544/276

(57) ABSTRACT

Compounds that inhibit PI3Kδ activity, including compounds that selectively inhibit PI3Kδ activity, are disclosed. Methods of inhibiting phosphatidylinositol 3-kinase delta isoform (PI3Kδ) activity, and methods of treating diseases, such as disorders of immunity and inflammation in which PI3Kδ plays a role in leukocyte function, using the compounds also are disclosed.

### FIELD OF THE INVENTION

The present invention relates generally to phosphatidylinositol 3-kinase (PI3K) enzymes, and more particularly to selective inhibitors of PI3Kδ activity and methods of using such inhibitors.

### BACKGROUND OF THE INVENTION

Cell signaling via 3′-phosphorylated phosphoinositides has been implicated in a variety of cellular processes, e.g., malignant transformation, growth factor signaling, inflammation, and immunity (see Rameh et al., J. Biol Chem, 274:8347-8350 (1999) for a review). The enzyme responsible for generating these phosphorylated signaling products is phosphatidylinositol 3-kinase (PI 3-kinase; PI3K). PI3K originally was identified as an activity associated with viral oncoproteins and growth factor receptor tyrosine kinases that phosphorylates phosphatidylinositol (PI) and its phosphorylated derivatives at the 3′-hydroxyl of the inositol ring (Panayotou et al., Trends Cell Biol 2:358-60 (1992)).

Levels of phosphatidylinositol-3,4,5-triphosphate (PIP3), the primary product of PI 3-kinase activation, increase upon treatment of cells with a variety of agonists. PI 3-kinase activation, therefore, is believed to be involved in a range of cellular responses including cell growth, differentiation, and apoptosis (Parker et al., Current Biology, 5:577-99 (1995); Yao et al., Science, 267:2003-05 (1995)). Though the downstream targets of phosphorylated lipids generated following PI 3-kinase activation have not been well characterized, emerging evidence suggests that pleckstrin-homology domain- and FYVE-finger domain-containing proteins are activated when binding to various phosphatidylinositol lipids (Sternmark et al., J Cell Sci, 112:4175-83 (1999); Lemmon et al., Trends Cell Biol, 7:237-42 (1997)). In vitro, some isoforms of protein kinase C (PKC) are directly activated by PIP3, and the PKC-related protein kinase, PKB, has been shown to be activated by PI 3-kinase (Burgering et al., Nature, 376:599-602 (1995)).

Presently, the PI 3-kinase enzyme family is divided into three classes, based on their substrate specificities. Class I PI3Ks can phosphorylate phosphatidylinositol (PI), phosphatidylinositol-4-phosphate, and phosphatidylinositol-4,5-biphosphate (PIP2) to produce phosphatidylinositol-3-phosphate (PIP), phosphatidylinositol-3,4-biphosphate, and phosphatidylinositol-3,4,5-triphosphate, respectively. Class II PI3Ks phosphorylate PI and phosphatidylinositol-4-phosphate, whereas Class III PI3Ks can only phosphorylate PI.

The initial purification and molecular cloning of PI 3-kinase revealed that it was a heterodimer consisting of p85 and p110 subunits (Otsu et al., Cell, 65:91-104 (1991); Hiles et al., Cell, 70:419-29 (1992)). Since then, four distinct Class I PI3Ks have been identified, designated PI3K α, β, δ, and γ, each consisting of a distinct 110 kDa catalytic subunit and a regulatory subunit. More specifically, three of the catalytic subunits, i.e., p110α, p110β, and p110γ, each interact with the same regulatory subunit, p85; whereas p110γ interacts with a distinct regulatory subunit, p101. As described below, the patterns of expression of each of these PI3Ks in human cells and tissues are also distinct. Though a wealth of information has been accumulated on the cellular functions of PI 3-kinases in general, the roles played by the individual isoforms are largely unknown.

Cloning of bovine p110α has been described. This protein was identified as related to the Saccharomyces cerevisiae protein: Vps34p, a protein involved in vacuolar protein processing. The recombinant p110α product was also shown to associate with p85α, to yield a PI3K activity in transfected COS-1 cells. See Hiles et al., Cell, 70, 419-29 (1992).

The cloning of a second human p110 isoform, designated p110β, is described in Hu et al., Mol Cell Biol, 13:7677-88 (1993). This isoform is said to associate with p85 in cells, and to be ubiquitously expressed, as p110β mRNA has been found in numerous human and mouse tissues, as well as in human umbilical vein endothelial cells, Jurkat human leukemic T cells, 293 human embryonic kidney cells, mouse 3T3 fibroblasts, HeLa cells, and NBT2 rat bladder carcinoma cells. Such wide expression suggests that the p110β isoform is broadly important in signaling pathways.

Identification of the p1106 isoform of PI 3-kinase is described in Chantry et al., J Biol Chem, 272:19236-41 (1997). It was observed that the human p110δ isoform is expressed in a tissue-restricted fashion. It is expressed at high levels in lymphocytes and lymphoid tissues, suggesting that the protein might play a role in PI 3-kinase-mediated signaling in the immune system. Details concerning the P110δ isoform also can be found in U.S. Pat. Nos. 5,858,753; 5,822,910; and 5,985,589, each incorporated herein by reference. See also, Vanhaesebroeck et al., Proc Natl Acad Sci USA, 94:4330-5 (1997), and International Publication No WO 97/46688.

In each of the PI3Kα, β, and δ subtypes, the p85 subunit acts to localize PI 3-kinase to the plasma membrane by the interaction of its SH2 domain with phosphorylated tyrosine residues (present in an appropriate sequence context) in target proteins (Rameh et al., Cell, 83:821-30 (1995)). Two isoforms of p85 have been identified, p85α, which is ubiquitously expressed, and p85β, which is primarily found in the brain and lymphoid tissues (Volinia et al., Oncogene, 7:789-93 (1992)). Association of the p85 subunit to the PI 3-kinase p110α, β, or δ catalytic subunits appears to be required for the catalytic activity and stability of these enzymes. In addition, the binding of Ras proteins also upregulates PI 3-kinase activity.

The cloning of p110γ revealed still further complexity within the PI3K family of enzymes (Stoyanov et al., Science, 269:690-93 (1995)). The p110γ isoform is closely related to p110α and p110β (45-48% identity in the catalytic domain), but as noted does not make use of p85 as a targeting subunit. Instead, p110γ contains an additional domain termed a “pleckstrin homology domain” near its amino terminus. This domain allows interaction of p110γ with the βγ subunits of heterotrimeric G proteins and this interaction appears to regulate its activity.

The p101 regulatory subunit for PI3Kgamma was originally cloned in swine, and the human ortholog identified subsequently (Krugmann et al., J Biol Chem, 274:17152-8 (1999)). Interaction between the N-terminal region of p101 with the N-terminal region of p110γ appears to be critical for the PI3Kγ activation through Gβγ mentioned above.

A constitutively active PI3K polypeptide is described in International Publication No. WO 96/25488. This publication discloses preparation of a chimeric fusion protein in which a 102-residue fragment of p85 known as the inter-SH2 (iSH2) region is fused through a linker region to the N-terminus of murine p110. The p85 iSH2 domain apparently is able to activate PI3K activity in a manner comparable to intact p85 (Klippel et al., Mol Cell Biol, 14:2675-85 (1994)).

Thus, PI 3-kinases can be defined by their amino acid identity or by their activity. Additional members of this growing gene family include more distantly related lipid and protein kinases including Vps34 TOR1, TOR2 of Saccharomyces cerevisiae (and their mammalian homologs such as FRAP and mTOR), the ataxia telangiectasia gene product (ATR), and the catalytic subunit of DNA-dependent protein kinase (DNA-PK). See generally, Hunter, Cell, 83:1-4 (1995).

PI 3-kinase also appears to be involved in a number of aspects of leukocyte activation. A p85-associated PI 3-kinase activity has been shown to physically associate with the cytoplasmic domain of CD28, which is an important costimulatory molecule for the activation of T-cells in response to antigen (Pages et al., Nature, 369:327-29 (1994); Rudd, Immunity, 4:527-34 (1996)). Activation of T cells through CD28 lowers the threshold for activation by antigen and increases the magnitude and duration of the proliferative response. These effects are linked to increases in the transcription of a number of genes including interleukin-2 (IL2), an important T cell growth factor (Fraser et al., Science, 251:313-16 (1991)). Mutation of CD28 such that it can no longer interact with PI 3-kinase leads to a failure to initiate IL2 production, suggesting a critical role for PI 3-kinase in T cell activation.

Specific inhibitors against individual members of a family of enzymes provide invaluable tools for deciphering functions of each enzyme. Two compounds, LY294002 and wortmannin, have been widely used as PI 3-kinase inhibitors. These compounds, however, are nonspecific PI3K inhibitors, as they do not distinguish among the four members of Class I PI 3-kinases. For example, the IC₅₀ values of wortmannin against each of the various Class I PI 3-kinases are in the range of 1-10 nM. Similarly, the IC₅₀ values for LY294002 against each of these PI 3-kinases is about 1 μM (Fruman et al., Ann Rev Biochem, 67:481-507 (1998)). Hence, the utility of these compounds in studying the roles of individual Class I PI 3-kinases is limited.

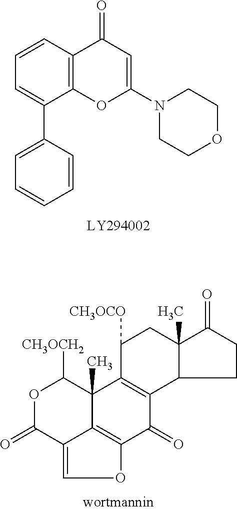

Based on studies using wortmannin, evidence exists that PI 3-kinase function also is required for some aspects of leukocyte signaling through G-protein coupled receptors (Thelen et al., Proc Natl Acad Sci USA, 91:4960-64 (1994)). Moreover, it has been shown that wortmannin and LY294002 block neutrophil migration and superoxide release. However, because these compounds do not distinguish among the various isoforms of PI3K, it remains unclear which particular PI3K isoform or isoforms are involved in these phenomena.

Recent publications demonstrate that selective inhibitors are known for PI3Kδ and that they are capable of treating certain types of disorders. Selective inhibitors of PI3Kδ are disclosed, for example, in U.S. Pat. Nos. 6,518,277; 6,667,300; 6,949,535; and 6,800,620, and in published U.S. Patent Application US 2006/0106038 and PCT applications WO 2005/113554, WO 2005/112935, and WO 2005/113556.

WO 2005/112935 discloses selective inhibitors of PI3Kδ, and indicates that they are useful to treat solid tumors such as carcinomas and sarcomas, as well as cancers involving vascular or lymphoreticular systems, lymphomas and hematological cancers such as myeloma and leukemia. It demonstrates that a selective inhibitor of PI3Kδ reduced tumor growth rates and vascularization significantly, and that when combined with a radiation treatment, the PI3Kδ inhibitor had a pronounced synergistic effect for reducing tumor vasculature development. Thus the compounds of the invention are useful to treat tumors by inhibiting angiogenesis, and they can be combined with other tumor treatments to provide a synergistic effect.

Puri, Current Enz Inhibition, 2, 147-61 (2006) discloses inhibition of acute myeloyid leukemia cell proliferation by a selective PI3Kδ inhibitor, without affecting proliferation of normal hematopoietic cells. It also describes evidence that such inhibitors are effective in animal models of hypertension.

Lee, et al., FASEB J. 20, 455-65 (2006) describes evidence that inhibition of PI3Kδ attenuates allergic airway inflammation and hyperrosponsiveness in murine asthma models, demonstrating that selective inhibitors of PI3Kδ are useful to treat asthma and allergic reactions as well as immune disorders. Billottet, et al., Oncogene 1-12 (2006) reports that a small molecule inhibitor of PI3Kδ inhibited cell proliferation in acute myeloid leukemia (AML) cultures and enhanced the cytotoxic effect of a widely used AML chemotherapy agent VP16 on AML cells. Thus it demonstrates that selective PI3Kδ inhibitors are useful to treat hematopoietic cancers and are synergistic with other therapeutic agents.

While some selective PI3Kδ inhibitors are thus known, a need remains for additional therapeutic agents useful to treat proliferative disorders, such as cancer, and excessive or destructive immune reactions, such as asthma, rheumatoid arthritis, multiple sclerosis, and lupus. The present invention provides novel compounds that are potent inhibitors of PI3Kδ, and are highly selective for the delta isoform and much less active against other isoforms of PI3K. These compounds are useful for the treatment of disorders associated with excessive activity, accumulation or production of hematopoietic cells, especially lymphocytes and leukocytes, including lymphomas, leukemias, and excessive immune response disorders.

### SUMMARY OF EMBODIMENTS

One aspect of the present invention is to provide compounds that can inhibit the biological activity of human PI3Kδ. Another aspect of the present invention is to provide compounds that inhibit PI3Kδ selectively compared to the other PI3K isoforms and that have a good bioavailability. Still another aspect of the invention is to provide a method of selectively modulating human PI3Kδ activity, and thereby promote medical treatment of diseases mediated by PI3Kδ function or dysfunction. Yet another aspect of the invention is to provide a method of characterizing the function of human PI3Kδ.

Another aspect of the present invention is to provide a method of disrupting leukocyte function comprising contacting leukocytes with a compound that selectively inhibits phosphatidylinositol 3-kinase delta (PI3Kδ) activity in the leukocytes. The leukocytes can comprise cells selected from the group consisting of neutrophils, B lymphocytes, T lymphocytes, and basophils.

For example, in cases wherein the leukocytes comprise neutrophils, the method comprises disrupting at least one neutrophil function selected from the group consisting of stimulated superoxide release, stimulated exocytosis, and chemotactic migration. Preferably, the method does not substantially disrupt bacterial phagocytosis or bacterial killing by the neutrophils. In cases wherein the leukocytes comprise B lymphocytes, the method comprises disrupting proliferation of the B lymphocytes or antibody production by the B lymphocytes. In cases wherein the leukocytes comprise T lymphocytes, the method comprises disrupting proliferation of the T lymphocytes. In cases wherein the leukocytes comprise basophils, the method comprises disrupting histamine release by the basophils.

In the present method, it is preferred that the PI3Kδ inhibitor is selective. It is preferred that the PI3Kδ inhibitor is at least about 10-fold selective for inhibition of PI3Kδ relative to other Type I PI3K isoforms in a biochemical assay. Preferably, the compound is at least about 20-fold selective, and more preferably, 30-fold selective, for inhibition of PI3Kδ relative to other Type I PI3K isoforms in a biochemical assay. In several embodiments, the compound is at least about 50-fold selective for inhibition of PI3Kδ relative to PI3Kα in a biochemical assay.

Compounds of the present invention are capable of inhibiting PI3Kδ activity and have a structural formula (I):

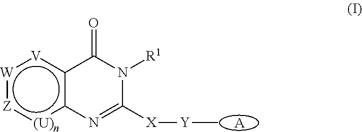

wherein U, V, W, and Z, independently, are selected from the group consisting of CR^{a}, N, NR^{b}, and O,

or wherein at least one of U, V, W and Z is N, and the others of U, V, W and Z are selected from the group consisting of CR^{a}, NR^{b}, S, and O,

and wherein at least one, but not all, of U, V, W, and Z is different from CR^{a};

A is an optionally substituted monocyclic or bicyclic ring system containing at least two nitrogen atoms as ring memebers, and at least one ring of the system is aromatic;

X is selected from the group consisting of C(R^{c})₂, C(R^{c})₂C(R^{c})₂, CH₂CHR^{c}, CHR^{c}CHR^{c}, CHR^{c}CH₂, CH═C(R^{c}), C(R^{c})═C(R^{c}) and C(R^{c})═CH;

Y is selected from the group consisting of null (i.e., a bond), S, SO, SO₂, NH, N(R^{c}), O, C(═O), OC(═O), C(═O)O, and NHC(═O)CH₂S;

R¹ is selected from the group consisting of H, substituted or unsubstituted C₁₋₁₀alkyl, substituted or unsubstituted C₂₋₁₀alkenyl, substituted or unsubstituted C₂₋₁₀alkynyl, substituted or unsubstituted C₁₋₆perfluoroalkyl, substituted or unsubstituted C₃₋₈cycloalkyl, substituted or unsubstituted C₃₋₈heterocycloalkyl, substituted or unsubstituted C₁₋₄alkyleneC₃₋₈cycloalkyl, substituted or unsubstituted aryl, substituted or unsubstituted heteroaryl, substituted or unsubstituted arylC₁₋₄alkyleneOR^{c}, substituted or unsubstituted heteroarylC₁₋₄alkyleneN(R^{d})₂, substituted or unsubstituted heteroarylC₁₋₄alkyleneOR^{e}, substituted or unsubstituted C₁₋₃alkyleneheteroaryl, substituted or unsubstituted C₁₋₃alkylenearyl, substituted or unsubstituted arylC₁₋₆alkyl, arylC₁₋₄alkyleneN(R^{d})₂, C₁₋₄alkyleneC(═O)C₁₋₄alkylenearyl, C₁₋₄alkyleneC(═O)C₁₋₄alkyleneheteroaryl, C₁₋₄alkyleneC(═O)heteroaryl, C₁₋₄alkyleneC(═O)N(R^{d})₂, C₁₋₆alkyleneOR^{d}, C₁₋₄alkyleneNR^{a}C(═O)R^{d}, C₁₋₄alkyleneOC₁₋₄alkyleneOR^{d}, C₁₋₄akyleneN(R^{d})₂, C₁₋₄alkyleneC(═O)OR^{d}, and C₁₋₄alkyleneOC₁₋₄alkyleneC(═O)OR^{d};

R^{a}, independently, is selected from the group consisting of H, substituted or unsubstituted C₁₋₆alkyl, substituted or unsubstituted C₃₋₈cycloalkyl, substituted or unsubstituted C₃₋₈heterocycloalkyl, substituted or unsubstituted aryl, C₁₋₃alkylenearyl, substituted or unsubstituted heteroaryl, substituted or unsubstituted heteroarylC₁₋₃alkyl, substituted or unsubstituted C₁₋₃alkyleneheteroaryl, halo, NHC(═O)C₁₋₃alkyleneN(R^{d})₂, NO₂, OR^{e}, CF₃, OCF₃, N(R^{d})₂, CN, OC(═O)R^{d}, C(═O)R^{d}, C(═O)OR^{d}, arylOR^{e}, NR^{d}C(═O)C₁₋₃alkyleneC(═O)OR^{d}, arylOC₁₋₃alkyleneN(R^{d})₂, arylOC(═O)R^{d}, C₁₋₄alkyleneC(═O)OR^{d}, OC₁₋₄alkyleneC(═O)OR^{d}, C₁₋₄alkyleneOC₁₋₄alkyleneC(═O)OR^{d}, C(═O)NR^{d}SO₂R^{d}, C₁₋₄alkyleneN(R^{d})₂, C₂₋₆alkenyleneN(R^{d})₂, C(═O)NR^{d}C₁₋₄alkyleneOR^{e}, C(═O)NR^{d}C₁₋₄alkyleneheteroaryl, OC₁₋₄alkyleneN(R^{d})₂, OC₁₋₄alkyleneCH(OR^{e})CH₂N(R^{d})₂, OC₁₋₄alkyleneheteroaryl, OC₂₋₄alkyleneOR^{e}, OC₂₋₄alkyleneNR^{d}C(═O)OR^{d}, NR^{a}C₁₋₄alkyleneN(R^{d})₂, NR^{a}C═O)R^{d}, NR^{a}C(═O)N(R^{d})₂, N(SO₂C₁₋₄alkyl)₂, NR^{a}(SO₂C₁₋₄alkyl), SO₂N(R^{d})₂, OSO₂CF₃, C₁₋₃alkylenearyl, C₁₋₄alkyleneeteroaryl, C₁₋₆alkyleneOR^{e}, C(═O)N(R^{d})₂, NHC(═O)C₁₋₃alkylenearyl, arylOC₁₋₃alkyleneN(R^{d})₂, arylOC(═O)R^{d}, NHC(═O)C₁₋₃alkyleneC₃₋₈heterocycloalkyl, NHC(═O)C₁₋₃alkyleneheteroaryl, OC₁₋₄alkleneOC₁₋₄alkyleneC(═O)OR^{d}, C(═O)C₁₋₄alkyleneheteroaryl, and NHC(═O)haloC₁₋₆alkyl;

R^{b} is selected from the group consisting of null, H, substituted or unsubstituted C₁₋₆alkyl, substituted or unsubstituted C₃₋₈cycloalkyl, substituted or unsubstituted C₃₋₈heterocyclolkyl, substituted or unsubstituted aryl, substituted or unsubstituted arylC₁₋₃alkyl, C₁₋₃alkylenearyl, substituted or unsubstituted heteroaryl, heteroarylC₁₋₃alkyl, substituted or unsubstituted C₁₋₃alkyleneheteroaryl, C(═O)R^{d}, C(═O)OR^{d}, arylOR^{e}, arylOC₁₋₃alkyleneN(R^{d})₂, arylOC(═O)R^{d}, C₁₋₄alkyleneC(═O)OR^{d}, C₁₋₄alkyleneOC₁₋₄alkyleneC(═O)OR^{d}, C(═O)NR^{d}SO₂R^{d}, C₁₋₄alkyleneN(R^{d})₂, C₂₋₆alkenyleneN(R^{d})₂, C(═O)NR^{d}C₁₋₄alkyleneOR^{e}, C(═O)NR^{d}C₁₋₄alkyleneheteroaryl, SO₂N(R^{d})₂, C₁₋₃alkylenearyl, C₁₋₄alkyleneheteroaryl, C₁₋₆alkyleneOR^{e}, C₁₋₃alkyleneN(R^{d})₂, C(═O)N(R^{d})₂, arylOC₁₋₃alkyleneN(R^{d})₂, arylOC(═O)R^{d}, and C(═O)C₁₋₄alkyleneheteroaryl;

R^{c}, independently, is selected from the group consisting of H, substituted or unsubstituted C₁₋₁₀alkyl, substituted or unsubstituted C₃₋₈cycloalkyl, substituted or unsubstituted C₃₋₈heterocycloalkyl, substituted or unsubstituted C₁₋₄alkyleneN(R^{d})₂, substituted or unsubstituted C₁₋₃alkyleneheteroC₁₋₃alkyl, substituted or unsubstituted arylheteroC₁₋₃alkyl, substituted or unsubstituted aryl, substituted or unsubstituted heteroaryl, substituted or unsubstituted arylC₁₋₃alkyl, substituted or unsubstituted heteroarylC₁₋₃alkyl, C₁₋₃alkylenearyl, substituted or unsubstituted C₁₋₃alkyleneheteroaryl, C(═O)R^{d}, and C(═O)OR^{d},

or two R^{c} on the same atom or on adjacent connected atoms can cyclize to form a ring having 3-8 ring members, which ring is optionally substituted and may include up to two heteroatoms selected from NR^{d}, O and S as ring members;

R^{d} is selected from the group consisting of H, substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈heterocycloalkyl, substituted or unsubstituted C₁₋₃alkyleneN(R^{e})₂, aryl, substituted or unsubstituted arylC₁₋₃alkyl, substituted or unsubstituted C₁₋₃alkylenearyl, substituted or unsubstituted heteroaryl, substituted or unsubstituted heteroarylC₁₋₃alkyl, and substituted or unsubstituted C₁₋₃alkyleneheteroaryl;

or two R^{d} groups are taken together with the nitrogen to which they are attached to form a 5- or 6-membered ring, optionally containing a second heteroatom that is N, O or S;

R^{e} is selected from the group consisting of H, substituted or unsubstituted C₁₋₆alkyl, substituted or unsubstituted C₃₋₈cycloalkyl, substituted or unsubstituted aryl, and substituted or unsubstituted heteroaryl,

or two R^{e} groups are taken together with the nitrogen to which they are attached to form a 5- or 6-membered ring, optionally containing a second heteroatom that is N, O or S;

said A, R¹, R^{a}, R^{b}, R^{c}, and R^{d}, independently, are optionally substituted with one to three substituents selected from the group consisting of C₁₋₁₀alkyl, C₂₋₁₀alkenyl, C₂₋₁₀alkynyl, C₃₋₈cycloalkyl, C₃₋₈heterocycloalkyl, C₁₋₆alkyleneOR^{c}, C₁₋₄alkyleneN(R^{c})₂, aryl, C₁₋₃alkylenearyl, heteroaryl, C(═O)OR^{e}, C(═O)R^{e}, OC(═O)R^{e}, halo, CN, CF₃, NO₂, N(R^{e})₂, OR^{e}, OC₁₋₆perfluoroalkyl, OC(═O)N(R^{e})₂, C(═O)N(R^{e})₂, SR^{e}, SO₂R^{e}, SO₃R^{e}, oxo(═O), and CHO; and

n is 0 or 1; or

a pharmaceutically acceptable salt, or prodrug, or solvate (e.g., hydrate) thereof.

Another aspect of the present invention is to provide a method of treating a medical condition mediated by neutrophils comprising administering to a mammal in need thereof a therapeutically effective amount of a compound that selectively inhibits phosphatidylinositol 3-kinase delta (PI3Kδ) activity in the neutrophils. Exemplary medical conditions that can be treated according to the method include those conditions characterized by an undesirable neutrophil function selected from the group consisting of stimulated superoxide release, stimulated exocytosis, and chemotactic migration. Preferably, according to the method, phagocytic activity or bacterial killing by the neutrophils is substantially uninhibited.

Still another aspect of the present invention is to provide a method of disrupting a function of osteoclasts comprising contacting osteoclasts with a compound that selectively inhibits PI3Kδ activity in the osteoclasts. According to the method, the compound comprises a moiety that preferentially binds to bone.

Another aspect of the present invention is to provide a method of ameliorating a bone-resorption disorder in a mammal in need thereof comprising administering to the mammal a therapeutically effective amount of a compound that inhibits PI3Kδ activity in osteoclasts of the mammal. A preferred bone-resorption disorder amenable to treatment according to the method is osteoporosis.

Another aspect of the present invention is to provide a method of ameliorating an excessive or undesired immune response in a mammal in need thereof comprising administering to the mammal a therapeutically effective amount of a compound that inhibits PI3Kδ activity in osteoclasts of the mammal. Examples of excessive or undesired immune responses treatable with the compounds of formula (I) include but are not limited to asthma, rheumatoid arthritis, lupus erythematosus, and multiple sclerosis. Other such disorders include disorders such as autoimmune thyroiditis, multiple sclerosis, some forms of diabetes, and Reynaud's syndrome; transplant rejection disorders such as GVHD and allograft rejection; chronic glomerulonephritis; inflammatory bowel diseases, such as chronic inflammatory bowel disease (CIBD), Crohn's disease, ulcerative colitis, and necrotizing enterocolitis; inflammatory dermatoses, such as contact dermatitis, atopic dermatitis, psoriasis, or urticaria; fever and myalgias due to infection.

Yet another aspect of the present invention is to provide a method of inhibiting the growth or proliferation of cancer cells of hematopoietic origin comprising contacting the cancer cells with a compound that selectively inhibits PI3Kδ activity in the cancer cells. The method can be advantageous in inhibiting the growth or proliferation of cancers selected from the group consisting of lymphomas, multiple myelomas, and leukemias.

Another aspect of the present invention is to provide a method of inhibiting kinase activity of a PI3Kδ polypeptide comprising contacting the PI3Kδ polypeptide with a compound having a structural formula (I).

Still another aspect of the present invention is to provide a method of disrupting leukocyte function comprising contacting leukocytes with a compound having a structural formula (I).

Another aspect of the present invention is to provide compounds having a structural formula (I) that inhibit PI3Kδ activity in biochemical and cell-based assays, and exhibit a therapeutic benefit in treating medical conditions wherein PI3Kδ activity is excessive or undesirable.

These and other aspects and advantages of the present invention will become apparent from the following detailed description of selected embodiments, which are provided to enhance the understanding of the invention without limiting the scope of the invention.

### DETAILED DESCRIPTION OF SELECTED EMBODIMENTS

The present invention provides compounds that selectively inhibit the activity of PI3Kδ. The present invention further provides methods of inhibiting PI3Kδ activity, including methods of selectively modulating the activity of the PI3Kδ isozyme in cells, especially leukocytes, osteoclasts, and cancer cells. The methods include in vitro, in vivo, and ex vivo applications.

Of particular benefit are methods of selectively modulating PI3Kδ activity in the clinical setting to ameliorate diseases or disorders mediated by PI3Kδ activity. Thus, treatment of diseases or disorders characterized by excessive or inappropriate PI3Kδ activity can be treated through administration of selective modulators of PI3Kδ.

Moreover, the invention provides pharmaceutical compositions comprising a selective PI3Kδ inhibitor. Also provided are articles of manufacture comprising a selective PI3Kδ inhibitor compound (or a pharmaceutical composition comprising the compound) and instructions for using the compound. Other methods of the invention include enabling the further characterization of the physiological role of the isozyme.

The methods described herein benefit from the use of compounds that selectively inhibit, and preferably specifically inhibit, the activity of PI3Kδ in cells, including cells in vitro, in vivo, or ex vivo. Cells treated by methods of the present invention include those that express endogenous PI3Kδ, wherein endogenous indicates that the cells express PI3Kδ absent recombinant introduction into the cells of one or more polynucleotides encoding a PI3Kδ polypeptide or a biologically active fragment thereof. The present methods also encompass use of cells that express exogenous PI3Kδ, wherein one or more polynucleotides encoding PI3Kδ or a biologically active fragment thereof have been introduced into the cell using recombinant procedures.

The cells can be in vivo, i.e., in a living subject, e.g., a mammal, including humans, wherein a PI3Kδ inhibitor can be used therapeutically to inhibit PI3Kδ activity in the subject. Alternatively, the cells can be isolated as discrete cells or in a tissue, for ex vivo or in vitro methods. In vitro methods encompassed by the invention can comprise the step of contacting a PI3Kδ enzyme or a biologically active fragment thereof with an inhibitor compound of the invention. The PI3Kδ enzyme can include a purified and isolated enzyme, wherein the enzyme is isolated from a natural source (e.g., cells or tissues that normally express a PI3Kδ polypeptide absent modification by recombinant technology) or isolated from cells modified by recombinant techniques to express exogenous enzyme.

The term “selective PI3Kδ inhibitor” as used herein refers to a compound that inhibits the PI3Kδ isozyme more effectively than other isozymes of the PI3K family. A “selective PI3Kδ inhibitor” compound is understood to be more selective for PI3Kδ than compounds conventionally and generically designated PI3K inhibitors, e.g., wortmannin or LY294002. Concomitantly, wortmannin and LY294002 are deemed “nonselective PI3K inhibitors.” Moreover, compounds of the present invention selectively negatively regulate PI3Kδ expression or activity and possess acceptable pharmacological properties for use in the therapeutic methods of the invention.

The relative efficacies of compounds as inhibitors of an enzyme activity (or other biological activity) can be established by determining the concentrations at which each compound inhibits the activity to a predefined extent, then comparing the results. Typically, the preferred determination is the concentration that inhibits 50% of the activity in a biochemical assay, i.e., the 50% inhibitory concentration or “IC₅₀.” IC₅₀ determinations can be accomplished using conventional techniques known in the art. In general, an IC₅₀ can be determined by measuring the activity of a given enzyme in the presence of a range of concentrations of the inhibitor under study. The experimentally obtained values of enzyme activity then are plotted against the inhibitor concentrations used. The concentration of the inhibitor that shows 50% enzyme activity (as compared to the activity in the absence of any inhibitor) is taken as the IC₅₀ value. Analogously, other inhibitory concentrations can be defined through appropriate determinations of activity. For example, in some settings it can be desirable to establish a 90% inhibitory concentration, i.e., IC₉₀.

Compounds of the present invention exhibit an IC₅₀ value vs. PI3Kδ of about 10 μM or less. In several embodiments, the compounds have an IC₅₀ vs. PI3Kδ of less than 5 μM. In other embodiments, the compounds have an IC₅₀ value vs. PI3Kδ of less than 1 μm, for example, down to 1 nm

Accordingly, a “selective PI3Kδ inhibitor” alternatively can be understood to refer to a compound that exhibits a 50% inhibitory concentration (IC₅₀) with respect to PI3Kδ that is at least 10-fold, preferably at least 20-fold, and more preferably at least 30-fold, lower than the IC₅₀ value with respect to any or all of the other Class I PI3K family members. The term “specific PI3Kδ inhibitor” can be understood to refer to a selective PI3Kδ inhibitor compound that exhibits an IC₅₀ with respect to PI3Kδ that is at least 50-fold, preferably at least 100-fold, more preferably at least 200-fold, and still more preferably at least 500-fold, lower than the IC₅₀ with respect to any or all of the other PI3K Class I family members.

The most preferred compounds of the present invention, therefore, have a low IC₅₀ value vs. PI3Kδ (i.e., is a potent inhibitor), and are selective with respect to inhibiting PI3Kδ compared to the other PI3K isoforms.

In one embodiment, the present invention provides a method of inhibiting leukocyte function. More particularly, the present invention provides methods of inhibiting or suppressing functions of neutrophils and T and B lymphocytes. With respect to neutrophils, it unexpectedly has been found that inhibition of PI3Kδ activity inhibits functions of neutrophils. For example, it has been observed that the compounds of the present invention elicit inhibition of classical neutrophil functions, such as stimulated superoxide release, stimulated exocytosis, and chemotactic migration. However, it further has been observed that a method of the present invention permits suppression of certain functions of neutrophils, while not substantially affecting other functions of these cells. For example, it has been observed that phagocytosis of bacteria by neutrophils is not substantially inhibited by the selective PI3Kδ inhibitor compounds of the present invention.

Thus, the present invention includes methods of inhibiting neutrophil functions, without substantially inhibiting phagocytosis of bacteria. Neutrophil functions suitable for inhibition according to the present method include any function mediated by PI3Kδ activity or expression. Such functions include, without limitation, stimulated superoxide release, stimulated exocytosis or degranulation, chemotactic migration, adhesion to vascular endothelium (e.g., tethering/rolling of neutrophils, triggering of neutrophil activity, and/or latching of neutrophils to endothelium), transmural diapedesis, or emigration through the endothelium to peripheral tissues. In general, these functions can be collectively termed “inflammatory functions,” as they are typically related to neutrophil response to inflammation. The inflammatory functions of neutrophils can be distinguished from the bacterial killing functions exhibited by these cells, e.g., phagocytosis and killing of bacteria. Accordingly, the present invention further includes methods of treating disease states in which one or more of the inflammatory functions of neutrophils are abnormal or undesirable.

It further has been established that PI3Kδ plays a role in the stimulated proliferation of lymphocytes, including B cells and T cells. Moreover, PI3Kδ appears to play a role in stimulated secretion of antibodies by B cells. Selective PI3Kδ inhibitor compounds of the present invention have been employed to establish that these phenomena can be abrogated by inhibition of PI3Kδ. Thus, the present invention includes methods of inhibiting lymphocyte proliferation, and methods of inhibiting antibody production by B lymphocytes. Other methods enabled by the present invention include methods of treating disease states in which one or more of these lymphocyte functions are abnormal or undesirable.

It now has been determined that PI3Kδ activity can be inhibited selectively or specifically to facilitate treatment of a PI3Kδ-mediated disease, while reducing or eliminating complications that typically are associated with concomitant inhibition of the activity of other Class I PI 3-kinases. To illustrate this embodiment, methods of the invention can be practiced using members of a class of compounds that have been found to exhibit selective inhibition of PI3Kδ relative to other PI3K isoforms.

The methods of this invention can be practiced using compounds having a general structural formula (I). Preferred methods employ compounds that have been determined to exhibit at least a 10-fold selective inhibition of PI3Kδ relative to other PI3K isoforms.

For example, the methods can be practiced using the following compounds:

6-[1-(6-amino-purin-9-yl)-ethyl]-3-bromo-1-methyl-5-phenyl-1,5-dihydro-pyrazolo[3,4-d]pyrimidin-4-one;

3-bromo-1-methyl-5-phenyl-6-[(1-(9H-purin-6-ylsulfanyl)-ethyl]-1,5-dihydro-pyrazolo[3,4-d]pyrimidin-4-one;

3-methyl-5-phenyl-6-(9H-purin-6-ylsulfanylmethyl)-5H-isoxazolo[5,4-d]pyrimidin-4-one;

6-(6-amino-purin-6-ylmethyl)-3-methyl-5-phenyl-5H-isoxazolo[5,4-d]pyrimidin-4-one;

2-[1-(4-amino-benzoimidazol-1-yl)-ethyl]-3-phenyl-3H-pyrido[3,2-d]pyrimidin-4-one;

3-phenyl-2-[1-(9H-purin-6-ylamino)-ethyl]-3H-pyrido[3,2-d]-pyrimidin-4-one;

and mixtures thereof.

For compounds of structural formula (I) that have an asymmetric center, the methods can be practiced using a racemic mixture of the compounds or a specific enantiomer. The S enantiomer is sometimes preferred when X represents a chiral center, such as CHR^{c} where R^{c} is not H.

The methods of the invention can be practiced using members of a class of compounds that exhibit PI3Kδ inhibitory activity, thereby facilitating inhibition of PI3Kδ activity in diseases mediated thereby. In particular, the methods of the invention can be practiced using compounds having the general structural formula (I):

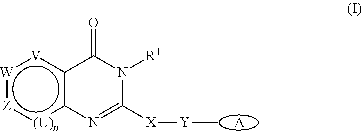

wherein U, V, W, and Z, independently, are selected from the group consisting of CR^{a}, N, NR^{b}, and O,

or wherein at least one of U, V, W and Z is N, and the others of U, V, W and Z are selected from the group consisting of CR^{a}, NR^{b}, S, and O,

and wherein at least one, but not all, of U, V, W, and Z is different from CR^{a};

A is an optionally substituted monocyclic or bicyclic ring system containing at least two nitrogen atoms as ring memebers, and at least one ring of the system is aromatic;

X is selected from the group consisting of C(R^{c})₂, C(R^{c})₂C(R^{c})₂, CH₂CHR^{c}, CHR^{c}CHR^{c}, CHR^{c}CH₂, CH═C(R^{c}), C(R^{c})═C(R^{c}) and C(R^{c})═CH;

Y is selected from the group consisting of null (i.e., a bond), S, SO, SO₂, NH, N(R^{c}), O, C(═O), OC(═O), C(═O)O, and NHC(═O)CH₂S;

R¹ is selected from the group consisting of H, substituted or unsubstituted C₁₋₁₀alkyl, substituted or unsubstituted C₂₋₁₀alkenyl, substituted or unsubstituted C₂₋₁₀alkynyl, substituted or unsubstituted C₁₋₆perfluoroalkyl, substituted or unsubstituted C₃₋₈cycloalkyl, substituted or unsubstituted C₃₋₈heterocycloalkyl, substituted or unsubstituted C₁₋₄alkyleneC₃₋₈cycloalkyl, substituted or unsubstituted aryl, substituted or unsubstituted heteroaryl, substituted or unsubstituted arylC₁₋₄alkyleneOR^{c}, substituted or unsubstituted heteroarylC₁₋₄alkyleneN(R^{d})₂, substituted or unsubstituted heteroarylC₁₋₄alkyleneOR^{e}, substituted or unsubstituted C₁₋₃alkyleneheteroaryl, substituted or unsubstituted C₁₋₃alkylenearyl, substituted or unsubstituted arylC₁₋₆alkyl, arylC₁₋₄alkyleneN(R^{d})₂, C₁₋₄alkyleneC(═O)C₁₋₄alkylenearyl, C₁₋₄alkyleneC(═O)C₁₋₄alkyleneheteroaryl, C₁₋₄alkyleneC(═O)heteroaryl, C₁₋₄alkyleneC(═O)N(R^{d})₂, C₁₋₆alkyleneOR^{d}, C₁₋₄alkyleneNR^{a}C(═O)R^{d}, C₁₋₄alkyleneOC₁₋₄alkyleneOR^{d}, C₁₋₄alkyleneN(R^{d})₂, C₁₋₄alkyleneC(═O)OR^{d}, and C₁₋₄alkyleneOC₁₋₄alkyleneC(═O)OR^{d};

R^{a}, independently, is selected from the group consisting of H, substituted or unsubstituted C₁₋₆alkyl, substituted or unsubstituted C₃₋₈cycloalkyl, substituted or unsubstituted C₃₋₈heterocycloalkyl, substituted or unsubstituted aryl, C₁₋₃alkylenearyl, substituted or unsubstituted heteroaryl, substituted or unsubstituted heteroarylC₁₋₃alkyl, substituted or unsubstituted C₁₋₃alkyleneheteroaryl, halo, NHC(═O)C₁₋₃alkyleneN(R^{d})₂, NO₂, OR^{e}, CF₃, OCF₃, N(R^{d})₂, CN, OC(═O)R^{d}, C(═O)R^{d}, C(═O)OR^{d}, arylOR^{e}, NR^{d}C(═O)C₁₋₃alkyleneC(═O)OR^{d}, arylOC₁₋₃alkyleneN(R^{d})₂, arylOC(═O)R^{d}, C₁₋₄alkyleneC(═O)OR^{d}, OC₁₋₄alkyleneC(═O)OR^{d}, C₁₋₄alkyleneOC₁₋₄alkyleneC(═O)OR^{d}, C(═O)NR^{d}SO₂R^{d}, C₁₋₄alkyleneN(R^{d})₂, C₂₋₆alkenyleneN(R^{d})₂, C(═O)NR^{d}C₁₋₄alkyleneOR^{e}, C(═O)NR^{d}C₁₋₄alkyleneheteroaryl, OC₁₋₄alkyleneN(R^{d})₂, OC₁₋₄alkyleneCH(OR^{e})CH₂N(R^{d})₂, OC₁₋₄alkyleneheteroaryl, OC₂₋₄alkyleneOR^{e}, OC₂₋₄alkyleneNR^{d}C(═O)OR^{d}, NR^{a}C₁₋₄alkyleneN(R)₂, NR^{a}C═O)R^{d}, NR^{a}C(═O)N(R^{d})₂, N(SO₂C₁₋₄alkyl)₂, NR^{a}(SO₂C₁₋₄alkl), SO₂N(R^{d})₂, OSO₂CF₃, C₁₋₃alkylenearyl, C₁₋₄alkyleneeteroaryl, C₁₋₆alkyleneOR^{e}, C(═O)N(R^{d})₂, NHC(═O)C₁₋₃alkylenearyl, arylOC₁₋₃alkyleneN(R^{d})₂, arylOC(═O)R^{d}, NHC(═O)C₁₋₃alkyleneC₃₋₈heterocycloalkyl, NHC(═O)C₁₋₃alkyleneheteroaryl, OC₁₋₄alkleneOC₁₋₄alkyleneC(═O)OR^{d}, C(═O)C₁₋₄alkyleneheteroaryl, and NHC(═O)haloC₁₋₆alkyl;

R^{b} is selected from the group consisting of null, H, substituted or unsubstituted C₁₋₆alkyl, substituted or unsubstituted C₃₋₈cycloalkyl, substituted or unsubstituted C₃₋₈heterocyclolkyl, substituted or unsubstituted aryl, substituted or unsubstituted arylC₁₋₃alkyl, C₁₋₃alkylenearyl, substituted or unsubstituted heteroaryl, heteroarylC₁₋₃alkyl, substituted or unsubstituted C₁₋₃alkyleneheteroaryl, C(═O)R^{d}, C(═O)OR^{d}, arylOR^{e}, arylOC₁₋₃alkyleneN(R^{d})₂, arylOC(═O)R^{d}, C₁₋₄alkyleneC(═O)OR^{d}, C₁₋₄alkyleneOC₁₋₄alkyleneC(═O)OR^{d}, C(═O)NR^{d}SO₂R^{d}, C₁₋₄alkyleneN(R^{d})₂, C₂₋₆alkenyleneN(R^{d})₂, C(═O)NR^{d}C₁₋₄alkyleneOR^{e}, C(═O)NR^{d}C₁₋₄alkyleneheteroaryl, SO₂N(R^{d})₂, C₁₋₃alkylenearyl, C₁₋₄alkyleneheteroaryl, C₁₋₆alkyleneOR^{e}, C₁₋₃alkyleneN(R^{d})₂, C(═O)N(R^{d})₂, arylOC₁₋₃alkyleneN(R^{d})₂, arylOC(═O)R^{d}, and C(═O)C₁₋₄alkyleneheteroaryl;

R^{c}, independently, is selected from the group consisting of H, substituted or unsubstituted C₁₋₁₀alkyl, substituted or unsubstituted C₃₋₈cycloalkyl, substituted or unsubstituted C₃₋₈heterocycloalkyl, substituted or unsubstituted C₁₋₄alkyleneN(R^{d})₂, substituted or unsubstituted C₁₋₃alkyleneheteroC₁₋₃alkyl, substituted or unsubstituted arylheteroC₁₋₃alkyl, substituted or unsubstituted aryl, substituted or unsubstituted heteroaryl, substituted or unsubstituted arylC₁₋₃alkyl, substituted or unsubstituted heteroarylC₁₋₃alkyl, C₁₋₃alkylenearyl, substituted or unsubstituted C₁₋₃alkyleneheteroaryl, C(═O)R^{d}, and C(═O)OR^{d},

or two R^{c} on the same atom or on adjacent connected atoms can cyclize to form a ring having 3-8 ring members, which ring is optionally substituted and may include up to two heteroatoms selected from NR^{d}, O and S as ring members;

R^{d} is selected from the group consisting of H, substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈heterocycloalkyl, substituted or unsubstituted C₁₋₃alkyleneN(R^{e})₂, aryl, substituted or unsubstituted arylC₁₋₃alkyl, substituted or unsubstituted C₁₋₃alkylenearyl, substituted or unsubstituted heteroaryl, substituted or unsubstituted heteroarylC₁₋₃alkyl, and substituted or unsubstituted C₁₋₃alkyleneheteroaryl;

or two R^{d} groups are taken together with the nitrogen to which they are attached to form a 5- or 6-membered ring, optionally containing a second heteroatom that is N, O or S;

R^{e} is selected from the group consisting of H, substituted or unsubstituted C₁₋₆alkyl, substituted or unsubstituted C₃₋₈cycloalkyl, substituted or unsubstituted aryl, and substituted or unsubstituted heteroaryl,

or two R^{e} groups are taken together with the nitrogen to which they are attached to form a 5- or 6-membered ring, optionally containing a second heteroatom that is N, O or S;

said A, R¹, R^{a}, R^{b}, R^{c}, and R^{d}, independently, are optionally substituted with one to three substituents selected from the group consisting of C₁₋₁₀alkyl, C₂₋₁₀alkenyl, C₂₋₁₀alkynyl, C₃₋₈cycloalkyl, C₃₋₈heterocycloalkyl, C₁₋₆alkyleneOR^{e}, C₁₋₄alkyleneN(R^{e})₂, aryl, C₁₋₃alkylenearyl, heteroaryl, C(═O)OR^{e}, C(═O)R^{e}, OC(═O)R^{e}, halo, CN, CF₃, NO₂, N(R^{e})₂, OR^{e}, OC₁₋₆perfluoroalkyl, OC(═O)N(R^{e})₂, C(═O)N(R^{e})₂, SR^{e}, SO₂R^{e}, SO₃R^{e}, oxo(═O), and CHO; and

n is 0 or 1; or

a pharmaceutically acceptable salt, or prodrug, or solvate (e.g., hydrate) thereof.

The compounds of the present invention are selective inhibitors of PI3Kδ activity. The compounds exhibit inhibition of PI3Kδ in biochemical assays, and selectively disrupt function of PI3Kδ-expressing cells in cell-based assays. As described herein, the present compounds have demonstrated an ability to inhibit certain functions in neutrophils and other leukocytes, as well as functions of osteoclasts.

As used herein, the term “alkyl” is defined as straight chained or branched hydrocarbon groups or cyclic hydrocarbon groups containing the indicated number of carbon atoms, typically methyl, ethyl, and straight chain and branched propyl and butyl groups, and cyclopropyl, cyclopentyl and cyclohexyl groups, as well as combination of straight chain, branched chain and cyclic groups, e.g., cyclopropylmethyl and norbornyl. The hydrocarbon group can contain up to 16 carbon atoms, preferably one to eight carbon atoms. The term “alkyl” includes cyclic, bicyclic, and “bridged alkyl,” i.e., a C₆-C₁₆ bicyclic or polycyclic hydrocarbon group, for example, norbornyl, adamantyl, bicyclo[2.2.2]octyl, bicyclo[2.2.1]heptyl, bicyclo[3.2.1]octyl, or decahydronaphthyl. The term “cycloalkyl” is defined as a cyclic C₃-C₈ hydrocarbon group, e.g., cyclopropyl, cyclobutyl, cyclohexyl, and cyclopentyl.

The term “alkenyl” is defined identically as “alkyl,” except the hydrocarbon groups contain at least one carbon-carbon double bond. The term “alkynyl” defined identically as “alkyl,” except the hydrocarbon groups contain at least one carbon-carbon triple bond. “Cycloalkenyl” is defined similarly to cycloalkyl, except at least one carbon-carbon double bond is present in the ring.

The term “perfluoroalkyl” is defined as an alkyl group wherein each hydrogen atom is replaced by fluorine.

The term “alkylene” is defined as an alkyl group having a substituent, for example, the term “C₁₋₃alkylenearyl” refers to an alkyl group containing one to three carbon atoms, and substituted with an aryl group. Similarly, “alkylene” when used without description of another group can refer to a divalent alkyl group, which can link two other structural features together, for example CH₂ and (CH₂)₃ are 1-carbon and 3-carbon alkylene groups.

The term “halo” or “halogen” is defined herein to include fluorine, bromine, chlorine, and iodine. Often, fluoro or chloro is preferred.

The term “haloalkyl” is defined herein as an alkyl group substituted with one or more halo substituents, either fluoro, chloro, bromo, iodo, or combinations thereof. Similarly, “halocycloalkyl” is defined as a cycloalkyl group having one or more halo substituents.

The term “aryl,” alone or in combination, is defined herein as a monocyclic or polycyclic aromatic group, preferably a monocyclic or bicyclic aromatic group, e.g., phenyl or naphthyl. Unless otherwise indicated, an “aryl” group can be unsubstituted or substituted, for example, with one or more, and in particular one to three, halo, alkyl, phenyl, hydroxyalkyl, alkoxy, alkoxyalkyl, haloalkyl, nitro, amino, alkylamino, acylamino, alkylthio, alkylsulfinyl, and alkylsulfonyl. Exemplary aryl groups include phenyl, naphthyl, biphenyl, tetrahydronaphthyl, chlorophenyl, fluorophenyl, aminophenyl, methylphenyl, methoxyphenyl, trifluoromethylphenyl, nitrophenyl, carboxyphenyl, and the like. The terms “arylC₁₋₆alkyl” and “heteroarylC₁₋₆alkyl” are defined as an aryl or heteroaryl group having a C₁₋₆alkyl substituent.

The term “heteroaryl” is defined herein as a monocyclic or bicyclic ring system containing one or two aromatic rings and containing at least one nitrogen, oxygen, or sulfur atom in an aromatic ring, and which can be unsubstituted or substituted, for example, with one or more, and in particular one to three, substituents, like halo, alkyl, hydroxy, hydroxyalkyl, alkoxy, alkoxyalkyl, haloalkyl, nitro, amino, alkylamino, acylamino, alkylthio, alkylsulfinyl, and alkylsulfonyl. Examples of heteroaryl groups include thienyl, furyl, pyridyl, oxazolyl, quinolyl, isoquinolyl, indolyl, triazolyl, isothiazolyl, isoxazolyl, imidazolyl, benzothiazolyl, pyrazinyl, pyrimidinyl, thiazolyl, and thiadiazolyl.

The term “C₃₋₈heterocycloalkyl” is defined as monocyclic ring system containing one or more heteroatoms selected from the group consisting of oxygen, nitrogen, and sulfur. A “C₃₋₈heterocycloalkyl” group also can contain an oxo group (═O) attached to the ring. Nonlimiting examples of “C₃₋₈heterocycloalkyl” groups include 1,3-dioxolane, 2-pyrazoline, pyrazolidine, pyrrolidine, piperazine, a pyrroline, 2H-pyran, 4H-pyran, morpholine, thiopholine, piperidine, 1,4-dithiane, and 1,4-dioxane.

The term “hydroxy” is defined as —OH.

The term “alkoxy” is defined as —OR, wherein R is C1-C8 alkyl, C2-C8 alkenyl or C2-C8 alkynyl; each alkyl, alkenyl and alkynyl group is optionally substituted.

The term “alkoxyalkyl” is defined as an alkyl group wherein a hydrogen has been replaced by an alkoxy group. The term “(alkylthio)alkyl” is defined similarly as alkoxyalkyl, except a sulfur atom, rather than an oxygen atom, is present.

The term “hydroxyalkyl” is defined as a hydroxy group appended to an alkyl group.

The term “amino” is defined as —NH₂, and the term “alkylamino” is defined as —NR₂, wherein at least one R is alkyl and the second R is alkyl or hydrogen.

The term “acylamino” is defined as RC(═O)N, wherein R is alkyl or aryl.

The term “alkylthio” is defined as —SR, wherein R is alkyl.

The term “alkylsulfinyl” is defined as R—SO, wherein R is alkyl.

The term “alkylsulfonyl” is defined as R—SO₂, wherein R is alkyl.

The term “amino” is defined as —NH₂, and the term “alkylamino” is defined as —NR₂, wherein at least one R is alkyl, alkenyl or alkynyl, and the second R is alkyl, alkenyl, alkynyl or hydrogen.

The term “acylamino” is defined as RC(═O)N, wherein R is alkyl, alkenyl, alkynyl or aryl, heteroaryl, or heterocylyl.

The term “nitro” is defined as —NO₂.

The term “trifluoromethyl” is defined as —CF₃.

The term “trifluoromethoxy” is defined as —OCF₃.

The term “cyano” is defined as —CN.

Alkyl, alkenyl and alkynyl groups are often substituted to the extent that such substitution makes sense chemically. Typical substituents include, but are not limited to, halo, ═O, ═N—CN, ═N—OR, ═NR, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R is optionally substituted with halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂, wherein each R′ is independently H, C1-C8 alkyl, C2-C8 heteroalkyl, C1-C8 acyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl or C5-C10 heteroaryl, each of which can be substituted by the substituents that are appropriate for the particular group.

Aryl and heteroaryl moieties may be substituted with a variety of substituents including C1-C8 alkyl, C2-C8 alkenyl, C2-C8 alkynyl, C5-C12 aryl, C1-C8 acyl, and heteroforms of these, each of which can itself be further substituted; other substituents for aryl and heteroaryl moieties include halo, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C2-C8 alkenyl, C2-C8 heteroalkenyl, C2-C8 alkynyl, C2-C8 heteroalkynyl, C6-C10 aryl, C5-C10 heteroaryl, C7-C12 arylalkyl, or C6-C12 heteroarylalkyl, and each R is optionally substituted as described above for alkyl groups. The substituent groups on an aryl or heteroaryl group may of course be further substituted with the groups described herein as suitable for each type of such substituents or for each component of the substituent. Thus, for example, an arylalkyl substituent may be substituted on the aryl portion with substituents described herein as typical for aryl groups, and it may be further substituted on the alkyl portion with substituents described herein as typical or suitable for alkyl groups.

‘Heteroforms’ as used herein refers to a modified alkyl, alkenyl, aryl, etc. wherein at least one heteroatom selected from N, O and S replaces at least one carbon atom in the hydrocarbon group being described.

In preferred embodiments of the compounds of formula (I), X is selected from the group consisting of CH₂, CH₂CH₂, CH═CH, CH(CH₃), CH(CH₂CH₃), CH₂CH(CH₃), and C(CH₃)₂. In further preferred embodiments, Y is selected from the group consisting of null (i.e., Y is absent, so it represents a bond between X and A), S, and NH.

When X contains a chiral carbon, such as when X is CH(CH₃) or CH(CH₂CH₃), it is often preferable to use the S-enantiomer of X. In other embodiments, the R-enantiomer may be used.

In some embodiments, X is CHR^{c} or CHR^{c}CHR^{c}, and Y is NR^{c}, and two of the R^{c} groups cyclize to form a ring. Where X is chiral in such embodiments, the S enantiomer is often preferred in certain embodiments, and in some embodiments the R enantiomer is preferred. In some such embodiments, —X—Y— taken together can represent a ring such as this:

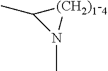

The A ring can be monocyclic or bicyclic. Monocyclic A ring systems are aromatic. Bicyclic A ring systems contain at least one aromatic ring, but both rings can be aromatic. Examples of A ring systems include, but are not limited to, imidazolyl, pyrazolyl, 1,2,3-triazolyl, pyridizinyl, pyrimidinyl, pyrazinyl, 1,3,5-triazinyl, purinyl, cinnolinyl, phthalazinyl, quinazolinyl, quinoxalinyl, 1,8-naphthyridinyl, pteridinyl, 1H-indazolyl, and benzimidazolyl. Some preferred embodiments of A comprise at least one pyrimidine ring, e.g., they include purine and pteridine ring systems as well as imidazolpyrimidines, pyrazolopyrimidines and pyrrolopyrimidines.

In some preferred compounds of structural formula (I), A is represented by an optionally substituted ring system selected from the group consisting of

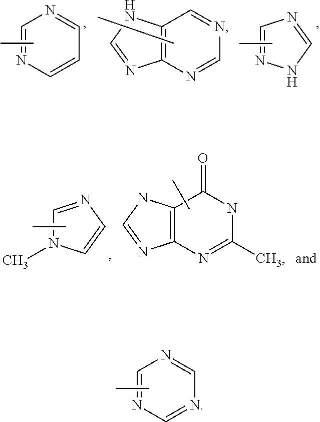

The A ring system optionally can be substituted with one to three, and preferably one or two, substituents selected from the group consisting of N(R^{e})₂, halo, C₁₋₃ haloalkyl, C₁₋₃alkyl, S(C₁₋₃alkyl), and OR^{c}. Specific substituents include, but are not limited to, NH₂, NH(CH₃), N(CH₃)₂, NHCH₂C₆H₅, NH(C₂H₅), Cl, F, CH₃, CF₃, SCH₃, and OH.

Especially preferred A rings include

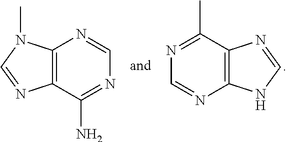

For the ring system

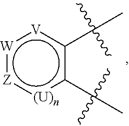

in preferred embodiments n is 0. Examples of preferred ring systems include, but are not limited to,

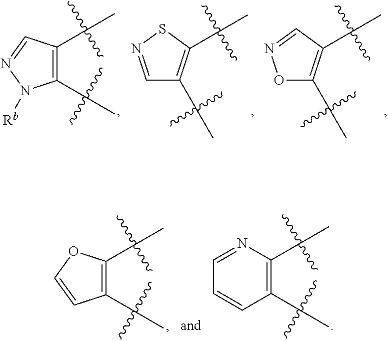

The ring systems are unsubstituted (i.e., R^{a} and R^{b} are hydro) or they may be substituted with substituents suitable for aryl or heteroaryl groups, preferably with one or more of C₁₋₆alkyl, halo, C₁₋₆alkoxy, CF₃, C₃₋₈cycloalkyl, aryl, or heteroaryl. Where the ring containing V, W and Z is substituted, it is often substituted by one or two substituents. In some embodiments, a substituent is on the atom represented by V, and in some embodiments it is on the atom represented by W.

In a preferred embodiment, R¹ in formula (I) is selected from the group consisting of optionally substituted C₁₋₆alkyl, aryl, heteroaryl, C₃₋₈cycloalkyl, C₃₋₈heterocycloalkyl, C₁₋₄alkyleneC₃₋₈heterocycloalkyl, C₁₋₄alkylenecycloalkyl, and C₁₋₄alkylenearyl. Specific R¹ groups include, but are not limited to, optionally substituted forms of:

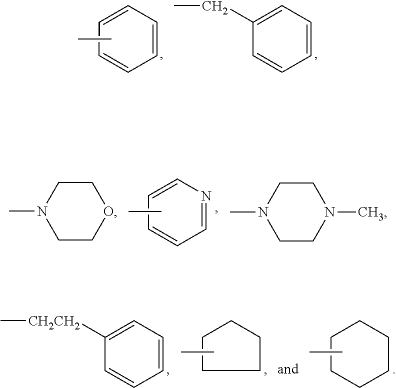

The R¹ group can be substituted with one to three substituents, for example, halo, OR^{e}, C₁₋₆alkyl, C₂₋₆alkenyl, C₂₋₆alkynyl, aryl, C₃₋₈heterocycloalkyl, heteroaryl, C₁₋₄alkyleneOR^{e}, CF₃, NO₂, N(R^{e})₂, C(═O)OR^{e}, SO₂N(R^{e})₂, CN, C(═O)R^{e}, C₁₋₄alkyleneN(R^{e})₂, OC₁₋₄perfluoroalkyl, oxo, and CHO. Specific substituents for the R¹ group include, but are not limited to, Cl, F, CH₃, CH(CH₃)₂, OH, OCH₃, (CH₂)₃N(CH₃)₂, CH₂C≡CH, C(═O)NH₂, C₆H₅, NO₂, NH₂, and CO₂H. In some embodiments, R¹ is preferably a phenyl group, heteroaryl group, or C₃₋₈ cycloalkyl or C₃₋₈ heterocycloalkyl, each of which is unsubstituted or is substituted with up to three substituents.

As used herein, the pyrimidin-4-one ring structure can be a 5,6-fused bicyclic or a 6,6-fused bicyclic system, and the following numbering of the ring structure is used for convenience:

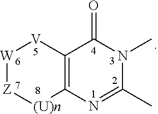

The purine ring structure is sometimes present as group A in formula (I) and for convenience the numbering of its ring structure, is

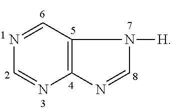

Where A represents purine, it is sometimes attached to Y at position 9 of the purine, and it is sometimes attached at position 6 of the purine. When A is attached to Y at position 6 of the purine ring, Y often represents S, NH or NR^{c}, and the purine group is often further substituted at position 9 by, for example, an amine.

It is generally accepted that biological systems can exhibit very sensitive activities with respect to the absolute stereochemical nature of compounds. See, E. J. Ariens, Medicinal Research Reviews, 6:451-466 (1986); E. J. Ariens, Medicinal Research Reviews, 7:367-387 (1987); K. W. Fowler, Handbook of Stereoisomers: Therapeutic Drugs, CRC Press, edited by Donald P. Smith, pp. 35-63 (1989); and S. C. Stinson, Chemical and Engineering News, 75:38-70 (1997).

Therefore, the present invention includes all possible stereoisomers and geometric isomers of compounds of structural formula (I) having an asymmetric center, and includes not only racemic compounds, but also the optically active isomers as well.

When a compound of structural formula (I) is desired as a single enantiomer, it can be obtained either by resolution of the final product or by stereospecific synthesis from either isomerically pure starting material or use of a chiral auxiliary reagent. For example, see Z. Ma et al., Tetrahedron: Asymmetry, 8(6), pages 883-888 (1997). Resolution of the final product, an intermediate, or a starting material can be achieved by any suitable method known in the art. Specific stereoisomers exhibit an excellent ability to inhibit kinase activity of PI3Kδ.

The term “prodrug” as used herein refers to compounds that are rapidly transformed in vivo to a compound having structural formula (I) or (II), for example, by hydrolysis. Prodrug design is discussed generally in Hardma et al. (Eds.), Goodman and Gilman's The Pharmacological Basis of Therapeutics, 9th ed., pp. 11-16 (1996). A thorough discussion of prodrugs is provided in Higuchi et al., Prodrugs as Novel Delivery Systems, Vol. 14, ASCD Symposium Series, and in Roche (ed.), “Bioreversible Carriers in Drug Design,” American Pharmaceutical Association and Pergamon Press (1987).

Briefly, administration of a drug is followed by elimination from the body or some biotransformation whereby biological activity of the drug is reduced or eliminated. Alternatively, a biotransformation process can lead to a metabolic by-product, which is itself more active or equally active as compared to the drug initially administered. Increased understanding of these biotransformation processes permits the design of so-called “prodrugs,” which, following a biotransformation, become more physiologically active in their altered state. Prodrugs, therefore, encompass pharmacologically inactive compounds that are converted to biologically active metabolites.

To illustrate, prodrugs can be converted into a pharmacologically active form through hydrolysis of, for example, an ester or amide linkage, thereby introducing or exposing a functional group on the resultant product. Prodrugs can be designed to react with an endogenous compound to form a water-soluble conjugate that further enhances the pharmacological properties of the compound, for example, increased circulatory half-life. Alternatively, prodrugs can be designed to undergo covalent modification on a functional group with, for example, glucuronic acid, sulfate, glutathione, amino acids, or acetate. The resulting conjugate can be inactivated and excreted in the urine, or rendered more potent than the parent compound. High molecular weight conjugates also can be excreted into the bile, subjected to enzymatic cleavage, and released back into the circulation, thereby effectively increasing the biological half-life of the originally administered compound.

### Methods for Identifying Negative Regulators of PI3Kδ Activity

The PI3Kδ protein, as well as fragments thereof possessing biological activity, can be used for screening putative negative regulator compounds in any of a variety of drug screening techniques. A negative regulator of PI3Kδ is a compound that diminishes or abolishes the ability of PI3Kδ to carry out any of its biological functions. An example of such compounds is an agent that decreases the ability of a PI3Kδ polypeptide to phosphorylate phosphatidylinositol or to target appropriate structures within a cell. The selectivity of a compound that negatively regulates PI3Kδ activity can be evaluated by comparing its activity on the PI3Kδ to its activity on other proteins. Selective negative regulators include, for example, antibodies and other proteins or peptides that specifically bind to a PI3Kδ polypeptide, oligonucleotides that specifically bind to PI3Kδ polypeptides, and other nonpeptide compounds (e.g., isolated or synthetic organic molecules) that specifically interact with PI3Kδ polypeptides. Negative regulators also include compounds as described above, but which interact with a specific binding partner of PI3Kδ polypeptides.

Presently preferred targets for the development of selective negative regulators of PI3Kδ include, for example:

(1) cytoplasmic regions of PI3Kδ polypeptides that contact other proteins and/or localize PI3Kδ within a cell;

(2) regions of PI3Kδ polypeptides that bind specific binding partners;

(3) regions of the PI3Kδ polypeptides that bind substrate;

(4) allosteric regulatory sites of the PI3Kδ polypeptides that can or cannot interact directly with the active site upon regulatory signal;

(5) regions of the PI3Kδ polypeptides that mediate multimerization.

For example, one target for development of modulators is the identified regulatory interaction of p85 with p110δ, which can be involved in activation and/or subcellular localization of the p110δ moiety. Still other selective modulators include those that recognize specific regulatory or PI3Kδ-encoding nucleotide sequences. Modulators of PI3Kδ activity can be therapeutically useful in treatment of a wide range of diseases and physiological conditions in which aberrant PI3Kδ activity is involved.

Accordingly, the invention provides methods of characterizing the potency of a test compound as an inhibitor of PI3Kδ polypeptide, said method comprising the steps of (a) measuring activity of a PI3Kδ polypeptide in the presence of a test compound; (b) comparing the activity of the PI3Kδ polypeptide in the presence of the test compound to the activity of the PI3Kδ polypeptide in the presence of an equivalent amount of a reference compound (e.g., a PI3Kδ inhibitor compound of the present invention, wherein a lower activity of the PI3Kδ polypeptide in the presence of the test compound than in the presence of the reference indicates that the test compound is a more potent inhibitor than the reference compound, and a higher activity of the PI3Kδ polypeptide in the presence of the test compound than in the presence of the reference indicates that the test compound is a less potent inhibitor than the reference compound.

The invention further provides methods of characterizing the potency of a test compound as an inhibitor of PI3Kδ polypeptide, comprising the steps of (a) determining an amount of a control compound (e.g., a PI3Kδ inhibitor compound of the present invention) that inhibits an activity of a PI3Kδ polypeptide by a reference percentage of inhibition, thereby defining a reference inhibitory amount for the control compound; (b) determining an amount of a test compound that inhibits an activity of a PI3Kδ polypeptide by a reference percentage of inhibition, thereby defining a reference inhibitory amount for the test compound; (c) comparing the reference inhibitory amount for the test compound to the reference inhibitory amount for the control compound, wherein a lower reference inhibitory amount for the test compound than for the control compound indicates that the test compound is a more potent inhibitor than the control compound, and a higher reference inhibitory amount for the test compound than for the control compound indicates that the test compound is a less potent inhibitor than the control compound. In one aspect, the method uses a reference inhibitory amount which is the amount of the compound than inhibits the activity of the PI3Kδ polypeptide by 50%, 60%, 70%, or 80%. In another aspect, the method employs a reference inhibitory amount that is the amount of the compound that inhibits the activity of the PI3Kδ polypeptide by 90%, 95%, or 99%. These methods comprise determining the reference inhibitory amount of the compounds in an in vitro biochemical assay, in an in vitro cell-based assay, or in an in vivo assay.

The invention further provides methods of identifying a negative regulator of PI3Kδ activity, comprising the steps of (i) measuring activity of a PI3Kδ polypeptide in the presence and absence of a test compound, and (ii) identifying as a negative regulator a test compound that decreases PI3Kδ activity and that competes with a compound of the invention for binding to PI3Kδ. Furthermore, the invention provides methods for identifying compounds that inhibit PI3Kδ activity, comprising the steps of (i) contacting a PI3Kδ polypeptide with a compound of the present invention in the presence and absence of a test compound, and (ii) identifying a test compound as a negative regulator of PI3Kδ activity wherein the compound competes with a compound of the invention for binding to PI3Kδ. The invention therefore provides a method of screening for candidate negative regulators of PI3Kδ activity and/or to confirm the mode of action of candidate such negative regulators. Such methods can be employed against other PI3K isoforms in parallel to establish comparative activity of the test compound across the isoforms and/or relative to a compound of the invention.

In these methods, the PI3Kδ polypeptide can be a fragment of p110δ that exhibits kinase activity, i.e., a fragment comprising the catalytic site of p110δ. Alternatively, the PI3Kδ polypeptide can be a fragment from the p110δ-binding domain of p85 and provides a method to identify allosteric modulators of PI3Kδ. The methods can be employed in cells expressing cells expressing PI3Kδ or its subunits, either endogenously or exogenously. Accordingly, the polypeptide employed in such methods can be free in solution, affixed to a solid support, modified to be displayed on a cell surface, or located intracellularly. The modulation of activity or the formation of binding complexes between the PI3Kδ polypeptide and the agent being tested then can be measured.

Human PI3K polypeptides are amenable to biochemical or cell-based high throughput screening (HTS) assays according to methods known and practiced in the art, including melanophore assay systems to investigate receptor-ligand interactions, yeast-based assay systems, and mammalian cell expression systems. For a review, see Jayawickreme et al., Curr Opin Biotechnol, 8:629-34 (1997). Automated and miniaturized HTS assays also are comprehended as described, for example, in Houston et al., Curr Opin Biotechnol, 8:734-40 (1997).

Such HTS assays are used to screen libraries of compounds to identify particular compounds that exhibit a desired property. Any library of compounds can be used, including chemical libraries, natural product libraries, and combinatorial libraries comprising random or designed oligopeptides, oligonucleotides, or other organic compounds. Chemical libraries can contain known compounds, proprietary structural analogs of known compounds, or compounds that are identified from natural product screening.

Natural product libraries are collections of materials isolated from naturals sources, typically, microorganisms, animals, plants, or marine organisms. Natural products are isolated from their sources by fermentation of microorganisms followed by isolation and extraction of the fermentation broths or by direct extraction from the microorganisms or tissues (plants or animal) themselves. Natural product libraries include polyketides, nonribosomal peptides, and variants (including nonnaturally occurring variants) thereof. For a review, see Cane et al., Science, 282:63-68 (1998).

Combinatorial libraries are composed of large numbers of related compounds, such as peptides, oligonucleotides, or other organic compounds as a mixture. Such compounds are relatively straightforward to design and prepare by traditional automated synthesis protocols, PCR, cloning, or proprietary synthetic methods. Of particular interest are peptide and oligonucleotide combinatorial libraries.

Still other libraries of interest include peptide, protein, peptidomimetic, multiparallel synthetic collection, recombinatorial, and polypeptide libraries. For a review of combinatorial chemistry and libraries created thereby, see Myers, Curr Opin Biotechnol, 8:701-07 (1997).

### Therapeutic Uses of Inhibitors of PI3Kδ Activity

The invention provides a method for selectively or specifically inhibiting PI3Kδ activity therapeutically or prophylactically. The method comprises administering a selective or specific inhibitor of PI3Kδ activity in an amount effective therefor. The method can be employed to treat humans or animals who are or can be subject to any condition whose symptoms or pathology is mediated by PI3Kδ expression or activity.

“Treating” as used herein refers to preventing a disorder from occurring in an animal that can be predisposed to the disorder, but has not yet been diagnosed as having it; inhibiting the disorder, i.e., arresting its development; relieving the disorder, i.e., causing its regression; or ameliorating the disorder, i.e., reducing the severity of symptoms associated with the disorder. “Disorder” is intended to encompass medical disorders, diseases, conditions, syndromes, and the like, without limitation.

The methods of the invention embrace various modes of treating an animal subject, preferably a mammal, more preferably a primate, and still more preferably a human. Among the mammalian animals that can be treated are, for example, companion animals (pets), including dogs and cats; farm animals, including cattle, horses, sheep, pigs, and goats; laboratory animals, including rats, mice, rabbits, guinea pigs, and nonhuman primates; and zoo specimens. Nonmammalian animals include, for example, birds, fish, reptiles, and amphibians.

A method of the present invention can be employed to treat subjects therapeutically or prophylactically who have or can be subject to an inflammatory disorder. One aspect of the present invention derives from the involvement of PI3Kδ in mediating aspects of the inflammatory process. Without intending to be bound by any theory, it is theorized that, because inflammation involves processes typically mediated by leukocyte (e.g., neutrophils or lymphocyte) activation and chemotactic transmigration, and because PI3Kδ can mediate such phenomena, antagonists of PI3Kδ can be used to suppress injury associated with inflammation.

“Inflammatory disorder” as used herein can refer to any disease, disorder, or syndrome in which an excessive or unregulated inflammatory response leads to excessive inflammatory symptoms, host tissue damage, or loss of tissue function. “Inflammatory disorder” also refers to a pathological state mediated by influx of leukocytes and/or neutrophil chemotaxis.

“Inflammation” as used herein refers to a localized, protective response elicited by injury or destruction of tissues, which serves to destroy, dilute, or wall off (sequester) both the injurious agent and the injured tissue. Inflammation is associated with an influx of leukocytes and/or neutrophil chemotaxis. Inflammation can result from infection with pathogenic organisms and viruses, and from noninfectious means such as trauma or reperfusion following myocardial infarction or stroke, immune response to foreign antigen, and autoimmune responses. Accordingly, inflammatory disorders amenable to the invention encompass disorders associated with reactions of the specific defense system as well as with reactions of the nonspecific defense system.

As used herein, the term “specific defense system” refers to the component of the immune system that reacts to the presence of specific antigens. Examples of inflammation resulting from a response of the specific defense system include the classical response to foreign antigens, autoimmune diseases, and delayed type hypersensitivity response mediated by T-cells. Chronic inflammatory diseases, the rejection of solid transplanted tissue and organs, e.g., kidney and bone marrow transplants, and graft versus host disease (GVHD), are further examples of inflammatory reactions of the specific defense system.

The term “nonspecific defense system” as used herein refers to inflammatory disorders that are mediated by leukocytes that are incapable of immunological memory (e.g., granulocytes, and macrophages). Examples of inflammation that result, at least in part, from a reaction of the nonspecific defense system include inflammation associated with conditions such as adult (acute) respiratory distress syndrome (ARDS) or multiple organ injury syndromes; reperfusion injury; acute glomerulonephritis; reactive arthritis; dermatoses with acute inflammatory components; acute purulent meningitis or other central nervous system inflammatory disorders such as stroke; thermal injury; inflammatory bowel disease; granulocyte transfusion associated syndromes; and cytokine-induced toxicity.

“Autoimmune disease” as used herein refers to any group of disorders in which tissue injury is associated with humoral or cell-mediated responses to the body's own constituents. “Allergic disease” as used herein refers to any symptoms, tissue damage, or loss of tissue function resulting from allergy. “Arthritic disease” as used herein refers to any disease that is characterized by inflammatory lesions of the joints attributable to a variety of etiologies. “Dermatitis” as used herein refers to any of a large family of diseases of the skin that are characterized by inflammation of the skin attributable to a variety of etiologies. “Transplant rejection” as used herein refers to any immune reaction directed against grafted tissue, such as organs or cells (e.g., bone marrow), characterized by a loss of function of the grafted and surrounding tissues, pain, swelling, leukocytosis, and thrombocytopenia.

The therapeutic methods of the present invention include methods for the treatment of disorders associated with inflammatory cell activation. “Inflammatory cell activation” refers to the induction by a stimulus (including, but not limited to, cytokines, antigens, or auto-antibodies) of a proliferative cellular response, the production of soluble mediators (including but not limited to cytokines, oxygen radicals, enzymes, prostanoids, or vasoactive amines), or cell surface expression of new or increased numbers of mediators (including, but not limited to, major histocompatability antigens or cell adhesion molecules) in inflammatory cells (including but not limited to monocytes, macrophages, T lymphocytes, B lymphocytes, granulocytes (i.e., polymorphonuclear leukocytes such as neutrophils, basophils, and eosinophils), mast cells, dendritic cells, Langerhans cells, and endothelial cells). It will be appreciated by persons skilled in the art that the activation of one or a combination of these phenotypes in these cells can contribute to the initiation, perpetuation, or exacerbation of an inflammatory disorder.

Compounds of the present invention have been found to inhibit superoxide release by neutrophils. Superoxide is released by neutrophils in response to any of a variety of stimuli, including signals of infection, as a mechanism of cell killing For example, superoxide release is known to be induced by tumor necrosis factor alpha (TNFα), which is released by macrophages, mast cells, and lymphocytes upon contact with bacterial cell wall components such as lipopolysaccharide (LPS). TNFα is an extraordinarily potent and promiscuous activator of inflammatory processes, being involved in activation of neutrophils and various other cell types, induction of leukocyte/endothelial cell adhesion, pyrexia, enhanced MHC class I production, and stimulation of angiogenesis. Alternatively, superoxide release can be stimulated by formyl-Met-Leu-Phe (fMLP) or other peptides blocked at the N-terminus by formylated methionine. Such peptides normally are not found in eukaryotes, but are fundamentally characteristic of bacteria, and signal the presence of bacteria to the immune system. Leukocytes expressing the fMLP receptor, e.g., neutrophils and macrophages, are stimulated to migrate up gradients of these peptides (i.e., chemotaxis) toward loci of infection. As demonstrated herein, compounds of the present invention inhibit stimulated superoxide release by neutrophils in response to either TNFα or fMLP. Other functions of neutrophils, including stimulated exocytosis and directed chemotactic migration, also have been shown to be inhibited by the PI3Kδ inhibitors of the invention. Accordingly, compounds of the present invention can be expected to be useful in treating disorders, such as inflammatory disorders, that are mediated by any or all of these neutrophil functions.

The present invention enables methods of treating such diseases as arthritic diseases, such as rheumatoid arthritis, monoarticular arthritis, osteoarthritis, gouty arthritis, spondylitis; Behcet disease; sepsis, septic shock, endotoxic shock, gram negative sepsis, gram positive sepsis, and toxic shock syndrome; multiple organ injury syndrome secondary to septicemia, trauma, or hemorrhage; ophthalmic disorders, such as allergic conjunctivitis, vernal conjunctivitis, uveitis, and thyroid-associated ophthalmopathy; eosinophilic granuloma; pulmonary or respiratory disorders, such as asthma, chronic bronchitis, allergic rhinitis, ARDS, chronic pulmonary inflammatory disease (e.g., chronic obstructive pulmonary disease), silicosis, pulmonary sarcoidosis, pleurisy, alveolitis, vasculitis, emphysema, pneumonia, bronchiectasis, and pulmonary oxygen toxicity; reperfusion injury of the myocardium, brain, or extremities; fibrosis, such as cystic fibrosis; keloid formation or scar tissue formation; atherosclerosis; autoimmune diseases, such as systemic lupus erythematosus (SLE), autoimmune thyroiditis, multiple sclerosis, some forms of diabetes, and Reynaud's syndrome; transplant rejection disorders such as GVHD and allograft rejection; chronic glomerulonephritis; inflammatory bowel diseases, such as chronic inflammatory bowel disease (CIBD), Crohn's disease, ulcerative colitis, and necrotizing enterocolitis; inflammatory dermatoses, such as contact dermatitis, atopic dermatitis, psoriasis, or urticaria; fever and myalgias due to infection; central or peripheral nervous system inflammatory disorders, such as meningitis, encephalitis, and brain or spinal cord injury due to minor trauma; Sjogren's syndrome; diseases involving leukocyte diapedesis; alcoholic hepatitis; bacterial pneumonia; antigen-antibody complex mediated diseases; hypovolemic shock; Type I diabetes mellitus; acute and delayed hypersensitivity; disease states due to leukocyte dyscrasia and metastasis; thermal injury; granulocyte transfusion-associated syndromes; and cytokine-induced toxicity.

The method can have utility in treating subjects who are or can be subject to reperfusion injury, i.e., injury resulting from situations in which a tissue or organ experiences a period of ischemia followed by reperfusion. The term “ischemia” refers to localized tissue anemia due to obstruction of the inflow of arterial blood. Transient ischemia followed by reperfusion characteristically results in neutrophil activation and transmigration through the endothelium of the blood vessels in the affected area. Accumulation of activated neutrophils in turn results in generation of reactive oxygen metabolites, which damage components of the involved tissue or organ. This phenomenon of “reperfusion injury” is commonly associated with conditions such as vascular stroke (including global and focal ischemia), hemorrhagic shock, myocardial ischemia or infarction, organ transplantation, and cerebral vasospasm. To illustrate, reperfusion injury occurs at the termination of cardiac bypass procedures or during cardiac arrest when the heart, once prevented from receiving blood, begins to reperfuse. It is expected that inhibition of PI3Kδ activity will result in reduced amounts of reperfusion injury in such situations.

With respect to the nervous system, global ischemia occurs when blood flow to the entire brain ceases for a period. Global ischemia can result from cardiac arrest. Focal ischemia occurs when a portion of the brain is deprived of its normal blood supply. Focal ischemia can result from thromboembolytic occlusion of a cerebral vessel, traumatic head injury, edema, or brain tumor. Even if transient, both global and focal ischemia can cause widespread neuronal damage. Although nerve tissue damage occurs over hours or even days following the onset of ischemia, some permanent nerve tissue damage can develop in the initial minutes following the cessation of blood flow to the brain.

Ischemia also can occur in the heart in myocardial infarction and other cardiovascular disorders in which the coronary arteries have been obstructed as a result of atherosclerosis, thrombus, or spasm. Accordingly, the invention is believed to be useful for treating cardiac tissue damage, particularly damage resulting from cardiac ischemia or caused by reperfusion injury in mammals.

In another aspect, selective PI3Kδ inhibitors of the present invention can be employed in methods of treating diseases of bone, especially diseases in which osteoclast function is abnormal or undesirable. As shown below, compounds of the present invention inhibit osteoclast function in vitro. Accordingly, the use of such compounds and other PI3Kδ selective inhibitors can be of value in treating osteoporosis, Paget's disease, and related bone resorption disorders.

In a further aspect, the present invention includes methods of using PI3Kδ inhibitory compounds to inhibit the growth or proliferation of cancer cells of hematopoietic origin, preferably cancer cells of lymphoid origin, and more preferably cancer cells related to or derived from B lymphocytes or B lymphocyte progenitors. Cancers amenable to treatment using the method of the invention include, without limitation, lymphomas, e.g., malignant neoplasms of lymphoid and reticuloendothelial tissues, such as Burkitt's lymphoma, Hodgkins' lymphoma, non-Hodgkins lymphomas, lymphocytic lymphomas and the like; multiple myelomas; leukemias, such as lymphocytic leukemias, chronic myeloid (myelogenous) leukemias, and the like. In a preferred embodiment, the present PI3Kδ inhibitory compounds can be used to inhibit or control the growth or proliferation of chronic myeloid (myelogenous) leukemia cells.

In another aspect, the invention includes a method of suppressing a function of basophils and/or mast cells, thereby enabling treatment of diseases or disorders characterized by excessive or undesirable basophil and/or mast cell activity. According to the method, a present compound can be used to selectively inhibit the expression or activity of PI3Kδ in the basophils and/or mast cells. Preferably, the method employs a PI3Kδ inhibitor in an amount sufficient to inhibit stimulated histamine release by the basophils and/or mast cells. Accordingly, the use of a present selective PI3Kδ inhibitors can be of value in treating diseases characterized by histamine release, i.e., allergic disorders, including disorders such as chronic obstructive pulmonary disease (COPD), asthma, ARDS, emphysema, and related disorders.

### Pharmaceutical Compositions of Inhibitors of PI3Kδ Activity

A compound of the present invention can be administered as the neat chemical, but it is typical, and preferable, to administer the compound in the form of a pharmaceutical composition or formulation. Accordingly, the present invention also provides pharmaceutical compositions that comprise a chemical or biological compound (“agent”) that is active as a modulator of PI3Kδ activity and a biocompatible pharmaceutical carrier, adjuvant, or vehicle. The composition can include the agent as the only active moiety or in combination with other agents, such as oligo- or polynucleotides, oligo- or polypeptides, drugs, or hormones mixed with excipient(s) or other pharmaceutically acceptable carriers. Carriers and other ingredients can be deemed pharmaceutically acceptable insofar as they are compatible with other ingredients of the formulation and not deleterious to the recipient thereof.

Techniques for formulation and administration of pharmaceutical compositions can be found in Remington's Pharmaceutical Sciences, 18th Ed., Mack Publishing Co, Easton, Pa., 1990. The pharmaceutical compositions of the present invention can be manufactured using any conventional method, e.g., mixing, dissolving, granulating, dragee-making, levigating, emulsifying, encapsulating, entrapping, melt-spinning, spray-drying, or lyophilizing processes. An optimal pharmaceutical formulation can be determined by one of skill in the art depending on the route of administration and the desired dosage. Such formulations can influence the physical state, stability, rate of in vivo release, and rate of in vivo clearance of the administered agent. Depending on the condition being treated, these pharmaceutical compositions can be formulated and administered systemically or locally.

The pharmaceutical compositions are formulated to contain suitable pharmaceutically acceptable carriers, and optionally can comprise excipients and auxiliaries that facilitate processing of the active compounds into preparations that can be used pharmaceutically. The administration modality will generally determine the nature of the carrier. For example, formulations for parenteral administration can comprise aqueous solutions of the active compounds in water-soluble form. Carriers suitable for parenteral administration can be selected from among saline, buffered saline, dextrose, water, and other physiologically compatible solutions. Preferred carriers for parenteral administration are physiologically compatible buffers such as Hank's solution, Ringer's solution, or physiologically buffered saline. For tissue or cellular administration, penetrants appropriate to the particular barrier to be permeated are used in the formulation. Such penetrants are generally known in the art. For preparations comprising proteins, the formulation can include stabilizing materials, such as polyols (e.g., sucrose) and/or surfactants (e.g., nonionic surfactants), and the like.

Alternatively, formulations for parenteral use can comprise dispersions or suspensions of the active compounds prepared as appropriate oily injection suspensions. Suitable lipophilic solvents or vehicles include fatty oils, such as sesame oil, and synthetic fatty acid esters, such as ethyl oleate or triglycerides, or liposomes. Aqueous injection suspensions can contain substances that increase the viscosity of the suspension, such as sodium carboxymethylcellulose, sorbitol, or dextran. Optionally, the suspension also can contain suitable stabilizers or agents that increase the solubility of the compounds to allow for the preparation of highly concentrated solutions. Aqueous polymers that provide pH-sensitive solubilization and/or sustained release of the active agent also can be used as coatings or matrix structures, e.g., methacrylic polymers, such as the EUDRAGIT® series available from Rohm America Inc. (Piscataway, N.J.). Emulsions, e.g., oil-in-water and water-in-oil dispersions, also can be used, optionally stabilized by an emulsifying agent or dispersant (surface active materials; surfactants). Suspensions can contain suspending agents such as ethoxylated isostearyl alcohols, polyoxyethlyene sorbitol and sorbitan esters, microcrystalline cellulose, aluminum metahydroxide, bentonite, agar-agar, gum tragacanth, and mixtures thereof.

Liposomes containing the active agent also can be employed for parenteral administration. Liposomes generally are derived from phospholipids or other lipid substances. The compositions in liposome form also can contain other ingredients, such as stabilizers, preservatives, excipients, and the like. Preferred lipids include phospholipids and phosphatidyl cholines (lecithins), both natural and synthetic. Methods of forming liposomes are known in the art. See, e.g., Prescott (Ed.), Methods in Cell Biology, Vol. XIV, p. 33, Academic Press, New York (1976).

Pharmaceutical compositions comprising the agent in dosages suitable for oral administration can be formulated using pharmaceutically acceptable carriers well known in the art. Preparations formulated for oral administration can be in the form of tablets, pills, capsules, cachets, dragees, lozenges, liquids, gels, syrups, slurries, elixirs, suspensions, or powders. To illustrate, pharmaceutical preparations for oral use can be obtained by combining the active compounds with a solid excipient, optionally grinding the resulting mixture, and processing the mixture of granules, after adding suitable auxiliaries if desired, to obtain tablets or dragee cores. Oral formulations can employ liquid carriers similar in type to those described for parenteral use, e.g., buffered aqueous solutions, suspensions, and the like.

Preferred oral formulations include tablets, dragees, and gelatin capsules. These preparations can contain one or excipients, which include, without limitation:

a) diluents, such as sugars, including lactose, dextrose, sucrose, mannitol, or sorbitol;

b) binders, such as magnesium aluminum silicate, starch from corn, wheat, rice, potato, etc.;

c) cellulose materials, such as methylcellulose, hydroxypropylmethyl cellulose, and sodium carboxymethylcellulose, polyvinylpyrrolidone, gums, such as gum arabic and gum tragacanth, and proteins, such as gelatin and collagen;

d) disintegrating or solubilizing agents such as cross-linked polyvinyl pyrrolidone, starches, agar, alginic acid or a salt thereof, such as sodium alginate, or effervescent compositions;

e) lubricants, such as silica, talc, stearic acid or its magnesium or calcium salt, and polyethylene glycol;

f) flavorants and sweeteners;

g) colorants or pigments, e.g., to identify the product or to characterize the quantity (dosage) of active compound; and

h) other ingredients, such as preservatives, stabilizers, swelling agents, emulsifying agents, solution promoters, salts for regulating osmotic pressure, and buffers.

Gelatin capsules include push-fit capsules made of gelatin, as well as soft, sealed capsules made of gelatin and a coating such as glycerol or sorbitol. Push-fit capsules can contain the active ingredient(s) mixed with fillers, binders, lubricants, and/or stabilizers, etc. In soft capsules, the active compounds can be dissolved or suspended in suitable fluids, such as fatty oils, liquid paraffin, or liquid polyethylene glycol with or without stabilizers.Dragee cores can be provided with suitable coatings such as concentrated sugar solutions, which also can contain gum arabic, talc, polyvinyl pyrrolidone, carbopol gel, polyethylene glycol, and/or titanium dioxide, lacquer solutions, and suitable organic solvents or solvent mixtures.

The pharmaceutical composition can be provided as a salt of the active agent. Salts are more soluble in aqueous or other protonic solvents than the corresponding free acid or base forms. Pharmaceutically acceptable salts are well known in the art. Compounds that contain acidic moieties can form pharmaceutically acceptable salts with suitable cations. Suitable pharmaceutically acceptable cations include, for example, alkali metal (e.g., sodium or potassium) and alkaline earth (e.g., calcium or magnesium) cations.

Compounds of structural formula (I) that contain basic moieties can form pharmaceutically acceptable acid addition salts with suitable acids. For example, Berge et al., J Pharm Sci, 66:1 (1977), describe pharmaceutically acceptable salts in detail. The salts can be prepared in situ during the final isolation and purification of the compounds of the invention or separately by reacting a free base function with a suitable acid.

Representative acid addition salts include, but are not limited to, acetate, adipate, alginate, citrate, aspartate, benzoate, benzenesulfonate, bisulfate, butyrate, camphorate, camphorolsulfonate, digluconate, glycerophosphate, hemisulfate, heptanoate, hexanoate, fumarate, hydrochloride, hydrobromide, hydroiodide, 2-hydroxyethanesulfonate (isothionate), lactate, maleate, methanesulfonate or sulfate, nicotinate, 2-naphthalenesulfonate, oxalate, pamoate, pectinate, persulfate, 3-phenylpropionate, picrate, pivalate, propionate, succinate, tartrate, thiocyanate, phosphate or hydrogen phosphate, glutamate, bicarbonate, p-toluenesulfonate, and undecanoate. Examples of acids that can be employed to form pharmaceutically acceptable acid addition salts include, without limitation, such inorganic acids as hydrochloric acid, hydrobromic acid, sulfuric acid, and phosphoric acid, and such organic acids as oxalic acid, maleic acid, succinic acid, and citric acid.

Basic addition salts can be prepared in situ during the final isolation and purification of the compounds of the invention or separately by reacting a carboxylic acid-containing moiety with a suitable base such as the hydroxide, carbonate, or bicarbonate of a pharmaceutically acceptable metal cation, or with ammonia or organic primary, secondary, or tertiary amine. Pharmaceutically acceptable basic addition salts include, but are not limited to, cations based on alkali metals or alkaline earth metals such as lithium, sodium, potassium, calcium, magnesium, and aluminum salts and the like, and nontoxic quaternary ammonium and amine cations including ammonium, tetramethylammonium, tetraethylammonium, methylammonium, dimethylammonium, trimethylammonium, ethylammonium, diethylammonium, triethylammonium, and the like. Other representative organic amines useful for the formation of base addition salts include ethylenediamine, ethanolamine, diethanolamine, piperidine, piperazine, and the like.

Basic nitrogen-containing groups can be quaternized with such agents as lower alkyl halides such as methyl, ethyl, propyl, and butyl chlorides, bromides and iodides; dialkyl sulfates like dimethyl, diethyl, dibutyl, and diamyl sulfates; long chain alkyl halides such as decyl, lauryl, myristyl, and stearyl chlorides, bromides, and iodides; arylalkyl halides such as benzyl and phenethyl bromides; and others. Products having modified solubility or dispersibility are thereby obtained.

In light of the foregoing, any reference to compounds of the present invention appearing herein is intended to include compounds of structural formula (I), as well as pharmaceutically acceptable salts, solvates, quaternary derivatives, and prodrugs, thereof.

Compositions comprising a compound of the invention formulated in a pharmaceutically acceptable carrier can be prepared, placed in an appropriate container, and labeled for treatment of an indicated condition. Accordingly, there also is contemplated an article of manufacture, such as a container comprising a dosage form of a compound of the invention and a label containing instructions for use of the compound. Kits also are contemplated. For example, a kit can comprise a dosage form of a pharmaceutical composition and a package insert containing instructions for use of the composition in treatment of a medical condition. In either case, conditions indicated on the label can include treatment of inflammatory disorders, cancer, and the like.

### Methods of Administration of Inhibitors of PI3Kδ Activity

Pharmaceutical compositions comprising an inhibitor of PI3Kδ activity can be administered to the subject by any conventional method, including parenteral and enteral techniques. Parenteral administration modalities include those in which the composition is administered by a route other than through the gastrointestinal tract, for example, intravenous, intraarterial, intraperitoneal, intramedullary, intramuscular, intraarticular, intrathecal, and intraventricular injections. Enteral administration modalities include, for example, oral (including buccal and sublingual) and rectal administration. Transepithelial administration modalities include, for example, transmucosal administration and transdermal administration. Transmucosal administration includes, for example, enteral administration as well as nasal, inhalation, and deep lung administration; vaginal administration; and rectal administration. Transdermal administration includes passive or active transdermal or transcutaneous modalities, including, for example, patches and iontophoresis devices, as well as topical application of pastes, salves, or ointments. Parenteral administration also can be accomplished using a high-pressure technique, e.g., POWDERJECT®.

Surgical techniques include implantation of depot (reservoir) compositions, osmotic pumps, and the like. A preferred route of administration for treatment of inflammation can be local or topical delivery for localized disorders such as arthritis, or systemic delivery for distributed disorders, e.g., intravenous delivery for reperfusion injury or for systemic conditions such as septicemia. For other diseases, including those involving the respiratory tract, e.g., chronic obstructive pulmonary disease, asthma, and emphysema, administration can be accomplished by inhalation or deep lung administration of sprays, aerosols, powders, and the like.

For the treatment of neoplastic diseases, especially leukemias and other distributed cancers, parenteral administration is typically preferred. Formulations of the compounds to optimize them for biodistribution following parenteral administration would be desirable. The PI3Kδ inhibitor compounds can be administered before, during, or after administration of chemotherapy, radiotherapy, and/or surgery.

Moreover, the therapeutic index of the PI3Kδ inhibitor compounds can be enhanced by modifying or derivatizing the compounds for targeted delivery to cancer cells expressing a marker that identifies the cells as such. For example, the compounds can be linked to an antibody that recognizes a marker that is selective or specific for cancer cells, so that the compounds are brought into the vicinity of the cells to exert their effects locally, as previously described (see for example, Pietersz et al., Immunol Rev, 129:57 (1992); Trail et al., Science, 261:212 (1993); and Rowlinson-Busza et al., Curr Opin Oncol, 4:1142 (1992)). Tumor-directed delivery of these compounds enhances the therapeutic benefit by, inter alia, minimizing potential nonspecific toxicities that can result from radiation treatment or chemotherapy. In another aspect, PI3Kδ inhibitor compounds and radioisotopes or chemotherapeutic agents can be conjugated to the same anti-tumor antibody.

For the treatment of bone resorption disorders or osteoclast-mediated disorders, the PI3Kδ inhibitors can be delivered by any suitable method. Focal administration can be desirable, such as by intraarticular injection. In some cases, it can be desirable to couple the compounds to a moiety that can target the compounds to bone. For example, a PI3Kδ inhibitor can be coupled to compounds with high affinity for hydroxyapatite, which is a major constituent of bone. This can be accomplished, for example, by adapting a tetracycline-coupling method developed for targeted delivery of estrogen to bone (Orme et al., Bioorg Med Chem Lett, 4(11):1375-80 (1994)).

To be effective therapeutically in modulating central nervous system targets, the agents used in the methods of the invention should readily penetrate the blood brain barrier when peripherally administered. Compounds that cannot penetrate the blood brain barrier, however, can still be effectively administered by an intravenous route.

As noted above, the characteristics of the agent itself and the formulation of the agent can influence the physical state, stability, rate of in vivo release, and rate of in vivo clearance of the administered agent. Such pharmacokinetic and pharmacodynamic information can be collected through preclinical in vitro and in vivo studies, later confirmed in humans during the course of clinical trials. Thus, for any compound used in the method of the invention, a therapeutically effective dose can be estimated initially from biochemical and/or cell-based assays. Then, dosage can be formulated in animal models to achieve a desirable circulating concentration range that modulates PI3Kδ expression or activity. As human studies are conducted, further information will emerge regarding the appropriate dosage levels and duration of treatment for various diseases and conditions.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the “therapeutic index,” which typically is expressed as the ratio LD₅₀/ED₅₀. Compounds that exhibit large therapeutic indices, i.e., the toxic dose is substantially higher than the effective dose, are preferred. The data obtained from such cell culture assays and additional animal studies can be used in formulating a range of dosage for human use. The dosage of such compounds lies preferably within a range of circulating concentrations that include the ED₅₀ with little or no toxicity.

In accordance with the present invention, any effective administration regimen regulating the timing and sequence of doses can be used. Doses of the agent preferably include pharmaceutical dosage units comprising an effective amount of the agent. As used herein, “effective amount” refers to an amount sufficient to modulate PI3Kδ expression or activity and/or derive a measurable change in a physiological parameter of the subject through administration of one or more of the pharmaceutical dosage units.

Exemplary dosage levels for a human subject are of the order of from about 0.001 milligram of active agent per kilogram body weight (mg/kg) to about 1000 mg/kg. Typically, dosage units of the active agent comprise from about 0.01 mg to about 1000 mg, preferably from about 0.1 mg to about 100 mg, depending upon the indication, route of administration, and severity of the condition, for example. Depending on the route of administration, a suitable dose can be calculated according to body weight, body surface area, or organ size. The final dosage regimen is determined by the attending physician in view of good medical practice, considering various factors that modify the action of drugs, e.g., the specific activity of the compound, the identity and severity of the disease state, the responsiveness of the patient, the age, condition, body weight, sex, and diet of the patient, and the severity of any infection. Additional factors that can be taken into account include time and frequency of administration, drug combinations, reaction sensitivities, and tolerance/response to therapy. Further refinement of the dosage appropriate for treatment involving any of the formulations mentioned herein is done routinely by the skilled practitioner without undue experimentation, especially in light of the dosage information and assays disclosed, as well as the pharmacokinetic data observed in human clinical trials. Appropriate dosages can be ascertained through use of established assays for determining concentration of the agent in a body fluid or other sample together with dose response data.

The frequency of dosing depends on the pharmacokinetic parameters of the agent and the route of administration. Dosage and administration are adjusted to provide sufficient levels of the active moiety or to maintain the desired effect. Accordingly, the pharmaceutical compositions can be administered in a single dose, multiple discrete doses, continuous infusion, sustained release depots, or combinations thereof, as required to maintain desired minimum level of the agent. Short-acting pharmaceutical compositions (i.e., short half-life) can be administered once a day or more than once a day (e.g., two, three, or four times a day). Long acting pharmaceutical compositions might be administered every 3 to 4 days, every week, or once every two weeks. Pumps, such as subcutaneous, intraperitoneal, or subdural pumps, can be used for continuous infusion.

The following examples are provided to further aid in understanding the invention, and presuppose an understanding of conventional methods well known to those persons having ordinary skill in the art to which the examples pertain, e.g., the construction of vectors and plasmids, the insertion of genes encoding polypeptides into such vectors and plasmids, or the introduction of vectors and plasmids into host cells. Such methods are described in detail in numerous publications including, for example, Sambrook et al., Molecular Cloning: A Laboratory Manual, Cold Spring Harbor Laboratory Press (1989), Ausubel et al. (Eds.), Current Protocols in Molecular Biology, John Wiley & Sons, Inc. (1994); and Ausubel et al. (Eds.), Short Protocols in Molecular Biology, 4th ed., John Wiley & Sons, Inc. (1999). The particular materials and conditions described hereunder are intended to exemplify particular aspects of the invention and should not be construed to limit the reasonable scope thereof.

### Example 1

Preparation and Purification of Recombinant PI3Kα, β, and δ

Recombinant PI3K heterodimeric complexes consisting of a p110 catalytic subunit and a p85 regulatory subunit were overexpressed using the BAC-TO-BAC® HT baculovirus expression system (GIBCO/BRL), and then purified for use in biochemical assays. The four Class I PI 3-kinases were cloned into baculovirus vectors as follows:

p110δ: A FLAG-tagged version of human p110δ (SEQ ID NO:1) (see Chantry et al., J Biol Chem, 272:19236-41 (1997)) was subcloned using standard recombinant DNA techniques into the BamH1-Xba1 site of the insect cell expression vector pFastbac HTb (Life Technologies, Gaithersburg, Md.), such that the clone was in frame with the His tag of the vector. The FLAG® system is described in U.S. Pat. Nos. 4,703,004; 4,782,137; 4,851,341; and 5,011,912, and reagents are available from Eastman Kodak Co.

p110α: Similar to the method used for p110δ, described above, a FLAG®-tagged version of p110α (see Volinia et al., Genomics, 24(3):427-477 (1994)) was subcloned in BamH1-HindIII sites of pFastbac HTb (Life Technologies) such that the clone was in frame with the His tag of the vector.

p110β: A p110β (see Hu et al., Mol Cell Biol, 13:7677-88 (1993)) clone was amplified from the human MARATHON® Ready spleen cDNA library (Clontech, Palo Alto Calif.) according to the manufacturer's protocol using the following primers:

5′ Primer
(SEQ ID NO: 3)
5′-GATCGAATTCGGCGCCACCATGGACTACAAGGACGACGATGACAA
GTGCTTCAGTTTCATAATGCCTCC-3′
3' Primer
(SEQ ID NO: 4)
5′-GATCGCGGCCGCTTAAGATCTGTAGTCTTTCCGAACTGTGTG-3′

The 5′ primer was built to contain a FLAG® tag in frame with the p110β sequence. After amplification, the FLAG®-p110β sequence was subcloned using standard recombinant techniques into the EcoR1-Not1 sites of pFastbac HTa (Life Technologies), such that the clone was in frame with the His tag of the vector.

p110γ: The p11oγ cDNA (see Stoyanov et al., Science, 269:690-93 (1995)) was amplified from a human Marathon Ready spleen cDNA library (Clontech) according to the manufacturer's protocol using the following primers:

5′ Primer
(SEQ ID NO: 5)
5′-AGAATGCGGCCGCATGGAGCTGGAGAACTATAAACAGCCC-3′
3′ Primer
(SEQ ID NO: 6)
5′-CGCGGATCCTTAGGCTGAATGTTTCTCTCCTTGTTTG-3′

A FLAG® tag was subsequently attached to the 5′ end of the p110γ sequence and was cloned in the BamH1-Spe1 sites of pFastbac HTb (Life Technologies) using standard recombinant DNA techniques, with the FLAG®-110γ sequence in-frame with the His tag of the vector.

p85α: A BamH1-EcoR1 fragment of FLAG®-tagged p85 cDNA (see Skolnik et al., Cell, 65:83-89 (1991)) was subcloned into the BamH1-EcoR1 sites of the vector pFastbac dual (Life Technologies).

Recombinant baculoviruses containing the above clones were generated using manufacturer's recommended protocol (Life Technologies). Baculoviruses expressing His-tagged p110α, p110β, or p110δ catalytic subunit and p85 subunit were coinfected into Sf21 insect cells. To enrich the heterodimeric enzyme complex, an excess amount of baculovirus expressing p85 subunit was infected, and the His-tagged p110 catalytic subunit complexed with p85 was purified on nickel affinity column. Since p110γ does not associate with p85, Sf21 cells were infected with recombinant baculoviruses expressing His-tagged p110γ only. In an alternate approach, p101 can be cloned into baculovirus, to permit coexpression with its preferred binding partner p110γ.

The 72-hour post-infected Sf21 cells (3 liters) were harvested and homogenized in a hypotonic buffer (20 mM HEPES-KOH, pH 7.8, 5 mM KCl, complete protease inhibitor cocktail (Roche Biochemicals, Indianapolis, Ind.), using a Dounce homogenizer. The homogenates were centrifuged at 1,000×g for 15 mM The supernatants were further centrifuged at 10,000×g for 20 mM, followed by ultracentrifugation at 100,000×g for 60 min. The soluble fraction was immediately loaded onto 10 mL of HITRAP® nickel affinity column (Pharmacia, Piscataway, N.J.) equilibrated with 50 mL of Buffer A (50 mM HEPES-KOH, pH 7.8, 0.5 M NaCl, 10 mM imidazole). The column was washed extensively with Buffer A, and eluted with a linear gradient of 10-500 mM imidazole. Free p85 subunit was removed from the column during the washing step and only the heterodimeric enzyme complex eluted at 250 mM imidazole. Aliquots of nickel fractions were analyzed by 10% SDS-polyacrylamide gel electrophoresis (SDS-PAGE), stained with SYPRO® Red (Molecular Probes, Inc., Eugene, Oreg.), and quantitated with STORM® Phospholmager (Molecular Dynamics, Sunnyvale, Calif.). The active fractions were pooled and directly loaded onto a 5 mL Hi-trap heparin column preequilibrated with Buffer B containing 50 mM HEPES-KOH, pH 7.5, 50 mM NaCl, 2 mM dithiothreitol (DTT). The column was washed with 50 mL of Buffer B and eluted with a linear gradient of 0.05-2 M NaCl. A single peak containing PI3K enzyme complex eluted at 0.8 M NaCl. SDS-polyacrylamide gel analysis showed that the purified PI3K enzyme fractions contained a 1:1 stoichiometric complex of p110 and p85 subunits. The protein profile of the enzyme complex during heparin chromatography corresponded to that of lipid kinase activity. The active fractions were pooled and frozen under liquid nitrogen.

### Example 2

PI3Kδ High Throughput Screen (HTS) and Selectivity Assay

A high throughput screen of a proprietary chemical library was performed to identify candidate inhibitors of PI3Kδ activity. PI3Kδ catalyzes a phosphotransfer from γ-[³²P]ATP to PIP₂/PS liposomes at the D3′ position of the PIP₂ lipid inositol ring. This reaction is MgCl₂ dependent and is quenched in high molarity potassium phosphate buffer pH 8.0 containing 30 mM EDTA. In the screen, this reaction is performed in the presence or absence of library compounds. The reaction products (and all unlabelled products) are transferred to a 96-well, prewetted PVDF filter plate, filtered, and washed in high molarity potassium phosphate. Scintillant is added to the dried wells and the incorporated radioactivity is quantitated.

The majority of assay operations were performed using a BIOMEK® 1000 robotics workstations (Beckman) and all plates were read using Wallac liquid scintillation plate counter protocols.

The 3× assay stocks of substrate and enzyme were made and stored in a trough (for robotics assays) or a 96-well, V-bottom, polypropylene plate (for manual assays). Reagents were stable for at least 3 hours at room temperature.

The 3× substrate for the HTS contained 0.6 mM Na₂ATP, 0.10 mCi/mL γ-[³²P]ATP (NEN, Pittsburgh, Pa.), 6 μM PIP₂/PS liposomes (Avanti Polar Lipids, Inc., Atlanta, Ga.), in 20 mM HEPES, pH 7.4.

The 3× enzyme stock for the HTS contained 1.8 nM PI3Kδ, 150 μg/mL horse IgG (used only as a stabilizer), 15 mM MgCl₂, 3 mM DTT in 20 mM HEPES, pH 7.4.

The chemical high throughput screen (HTS) library samples (each containing a pool of 22 compounds) in dimethyl sulfoxide (DMSO) were diluted to 18.75 μM or 37.8 μM in double distilled water, and 20 μL of the dilutions were placed in the wells of a 96-well polypropylene plate for assaying. The negative inhibitor control (or positive enzyme control) was DMSO diluted in water, and the positive inhibitor controls employed concentrations of LY294002 sufficient to provide 50% and 100% inhibition.

To the 20 μL pooled chemical library dilutions, 20 μL of 3× substrate was added. The reaction was initiated with 20 μL of 3× enzyme, incubated at room temperature for 10 minutes. This dilution established a final concentration of 200 μM ATP in the reaction volume. The reaction was stopped with 150 μL quench buffer (1.0 M potassium phosphate pH 8.0, 30 mM EDTA). A portion of the quenched solution (180 μL) then was transferred to a PVDF filter plate (Millipore #MAIP NOB prewetted with sequential 200 μL washes of 100% methanol, water, and finally 1.0 M potassium phosphate pH 8.0 wash buffer).

The PVDF filter plate was aspirated under moderate vacuum (2-5 mm Hg), washed with 5×200 μL of wash buffer, and then dried by aspiration. The filter was subsequently blotted, allowed to air dry completely, and inserted into a Wallac counting cassette with 50 μL of Ecoscint scintillation cocktail added per well. The incorporated radioactivity was quantitated, and data were analyzed, after normalizing to the enzyme positive control (set at 100%), to identify the curve intersection at the 50% inhibition value to estimate IC₅₀ values for the inhibitors. The inhibitors also were subjected to selectivity assays against PI3Kα and PI3Kδ (see assay protocol in Example 9).

From the selectivity assays, it was found that compounds of the present invention are potent and selective inhibitors of 3PIKδ. For example, as described above, the PI 3-kinase inhibitor LY294002 (Calbiochem, La Jolla, Calif.) does not have significant selectivity among the different PI 3-kinase isoforms tested. Under our assay conditions, LY294002 inhibited all three isoforms of PI 3-kinases with an IC₅₀ of 0.3 to 1 μM. The present compounds are at least 10 times less potent inhibitors of the α, β, and γ isoforms than the δ isoform. These results show that compounds of the present invention have the capability of selectively inhibiting PI3Kδ activity.

### Examples 3-7

Because PI3Kδ is expressed at significant levels in leukocytes, it is important to study the effects of the PI3Kδ-selective inhibitor on leukocyte functions. Accordingly, the effects of PI3Kδ inhibition in several types of leukocytes were examined. Neutrophils were examined to determine the effects that selective inhibition of PI3Kδ might elicit (Example 3, below). It surprisingly was found that selective inhibition of PI3Kδ activity appears to be significantly associated with inhibition of some but not all functions characteristic of activated neutrophils. In addition, the effects of PI3Kδ inhibition on B cell and T cell function also were tested (Examples 4-5, below). Moreover, as PI3Kδ also is expressed in osteoclasts, the effect of PI3Kδ inhibition on the function of these specialized cells was studied (Example 6, below). The effect of PI3Kδ on basophil function also was studied (Example 7, below).

### Example 3

Characterization of Role of PI3Kδ in Neutrophil Function

The effects of a PI3Kδ inhibitor of the invention on neutrophil functions such as superoxide generation, elastase exocytosis, chemotaxis, and bacterial killing can be tested.

A. Preparation of Neutrophils from Human Blood

Aliquots (8 mL) of heparinized blood from healthy volunteers are layered on 3 mL cushions of 7.3% FICOLL® (Sigma, St. Louis, Mo.) and 15.4% HYPAQUE® (Sigma) and centrifuged at 900 rpm for 30 min at room temperature in a table top centrifuge (Beckman). The neutrophil-rich band just above the FICOLL®-HYPAQUE® cushion is collected and washed with Hanks' balanced salt solution (HBSS) containing 0.1% gelatin. Residual erythrocytes are removed by hypotonic lysis with 0.2% NaCl. The neutrophil preparation is washed twice with HBSS containing 0.1% gelatin and used immediately.

B. Measurement of Superoxide Production from Neutrophils

Superoxide generation is one of the hallmarks of neutrophil activation. A variety of activators potentiate superoxide generation by neutrophils. The effect of a present PI3Kδ inhibitor on superoxide generation by three different agonists: TNF1α, IgG, and fMLP, each representing separate classes of activator, is measured. Superoxide generated by the neutrophils is measured by monitoring a change in absorbance upon reduction of cytochrome C by modification of the method described by Green et al., (pp. 14.5.1-14.5.11 in Supp. 12, Curr Protocols Immunol (Eds., Colligan et al.) (1994)), as follows. Individual wells of a 96-well plate are coated overnight at 4° C. with 50 μL of 2 mg/mL solution of human fibrinogen or IgG. The wells are washed with PBS and the following reagents were added to each well: 50 μL of HBSS or superoxide dismutase (1 mg/mL), 50 μL of HBSS or TNF1α (50 ng/mL), 50 μL cytochrome C (2.7 mg/mL), and 100 μL of purified human neutrophil suspension (2×10⁶ cells/mL). The plate is centrifuged for 2 min at 200 rpm and absorbance at 550 nm was monitored for 2 hr. To measure the relative amounts of superoxide generated, values obtained from the superoxide dismutase-containing wells are subtracted from all, and normalized to the values obtained from the wells without any inhibitor.

Compounds of the present invention inhibit TNF-induced superoxide generation by neutrophils in a concentration dependent manner. In addition, superoxide generation induced by IgG was not significantly inhibited by compounds of the present invention.

The effect of compounds of the present invention on superoxide generation induced by another potent inducer, the bacterial peptide formylated-Met-Leu-Phe (fMLP), also can be studied. Like the TNF-induced superoxide generation, fMLP-induced superoxide generation also is inhibited compounds of the present invention. These results show that the PI3Kδ inhibitor compounds of the present invention can prevent stimulus specific induction of superoxide generation by neutrophils, indicating that PI3Kδ is involved in this process.

C. Measurement of Elastase Exocytosis from Neutrophils

In addition to superoxide generation, activated neutrophils also respond by releasing several proteases that are responsible for the destruction of tissues and cartilage during inflammation. As an indication of protease release, the effect of present compound on elastase exocytosis is measured. Elastase exocytosis is quantitated by modification of the procedure described by Ossanna et al. (J Clin Invest, 77:1939-1951 (1986)), as follows. Purified human neutrophils (0.2×10⁶) (treated with either DMSO or a serial dilution of a present compound in DMSO) are stimulated with fMLP in PBS containing 0.01 mg/mL cytochalasin B, 1.0 μM sodium azide (NaN₃), 5 μg/mL L-methionine and 1 μM fMLP for 90 mM at 37° C. in a 96-well plate. At the end of the incubation period, the plate is centrifuged for 5 mM at 1000 rpm, and 90 μL of the supernatant is transferred to 10 μL of 10 mM solution of an elastase substrate peptide, MeO-suc-Ala-Ala-Pro-Val-pNA, wherein MeO-suc=methoxy-succinyl; pNA=p-nitroanilide (Calbiochem, San Diego, Calif.). Absorbance at 410 nm is monitored for 2 hr in a 96-well plate reader. To measure the relative amounts of elastase excytosed, all absorbance values are normalized to the values without any inhibitor. PI3Kδ inhibitor compounds of the present invention inhibit fMLP-induced elastase exocytosis significantly, and do so in a dose-dependent fashion.

D. Measurement of fMLP-Induced Human Neutrophil Migration

Neutrophils have the intrinsic capacity to migrate through tissues, and are one of the first cell types to arrive at the sites of inflammation or tissue injury. The effect of the present compounds on neutrophil migration towards a concentration gradient of fMLP is measured. The day before the migration assays are performed, 6-well plates are coated with recombinant ICAM-1/Fc fusion protein (Van der Vieren et al., Immunity, 3:683-690 (1995)) (25 μg/mL in bicarbonate buffer, pH 9.3) and left overnight at 4° C. After washing, 1% agarose solution, in RPMI-1640 with 0.5% bovine serum albumin (BSA), is added to wells with or without an inhibitor, and plates are placed into a refrigerator before punching holes in the gelled agarose to create plaques (1 central hole surrounded by 6 peripheral ones per well).

Human neutrophils are obtained as described above, and resuspended in RPMI medium supplemented with 0.5% BSA at 5×10⁶ cells/mL. After combining equal volumes of neutrophil suspension and medium (either with DMSO or a serial dilution of the test compound in DMSO), neutrophils are aliquoted into the peripheral holes, while the central hole received fMLP (5 μM). Plates are incubated at 37° C. in the presence of 5% CO₂ for 4 hr, followed by termination of migration by the addition of 1% glutaraldehyde solution in D-PBS. After removing the agarose layer, wells are washed with distilled water and dried.

Analysis of neutrophil migration is conducted on a Nikon DIAPHOT® inverted microscope (1× objective) video workstation using the NIH 1.61 program. Using Microsoft Excel and Table Curve 4 (SSPS Inc., Chicago Ill.) programs, a migration index is obtained for each of the studied conditions. Migration index is defined as the area under a curve representing number of migrated neutrophils versus the net distance of migration per cell.

PI3Kδ inhibitor compounds of the present invention have an effect on neutrophil migration, inhibiting this activity in a dose-dependent manner

E. Measurement of Bactericidal Capacity of Neutrophils

Given that the PI3Kδ inhibitor compounds of the present invention affect certain neutrophil functions, whether the compounds affect neutrophil-mediated bacterial killing is of interest. The effect of the compounds on neutrophil-mediated Staphylococcus aureus killing is studied according to the method described by Clark and Nauseef (pp. 7.23.4-7.23.6 in Vol. 2, Supp. 6, Curr Protocols Immunol (Eds., Colligan et al.) (1994)). Purified human neutrophils (5×10⁶ cells/mL) (treated with either DMSO or a serial dilution of present compound in DMSO) are mixed with autologous serum. Overnight-grown S. aureus cells are washed, resuspended in HBSS, and added to the serum-opsonized neutrophils at a 10:1 ratio. Neutrophils are allowed to internalize the bacteria by phagocytosis by incubation at 37° C. for 20 min. The noninternalized bacteria are killed by 10 units/mL lysostaphin at 37° C. for 5 min and the total mixture is rotated at 37° C. Samples are withdrawn at various times for up to 90 min and the neutrophils are lysed by dilution in water. Viable bacteria are counted by plating appropriate dilutions on trypticase-soy-agar plate and counting the S. aureus colonies after overnight growth.

Neutrophil-mediated killing of S. aureus is similar in samples treated with DMSO (control) and with a present compound. Therefore, a PI3Kδ inhibitor does not significantly affect the ability of neutrophils to kill S. aureus, suggesting that PI3Kδ is not involved in this pathway of neutrophil function.

### Example 4

Characterization of Role of PI3Kδ in B Lymphocyte Function

The effects of a PI3-kinase inhibitor on B cell functions including classical indices such as antibody production and specific stimulus-induced proliferation also are studied.

A. Preparation and Stimulation of B Cells from Peripheral Human Blood

Heparinized blood (200 mL) from healthy volunteers is mixed with an equal volume of D-PBS, layered on 10×10 mL FICOLL-PAQUE® (Pharmacia), and centrifuged at 1600 rpm for 30 min at room temperature. Peripheral blood mononuclear cells (PBMC) are collected from the FICOLL®/serum interface, overlayed on 10 mL fetal bovine serum (FBS) and centrifuged at 800 rpm for 10 min to remove platelets. After washing, cells are incubated with DYNAL® Antibody Mix (B cell kit) (Dynal Corp., Lake Success, N.Y.) for 20 min at 4-8° C. Following the removal of unbound antibody, PBL are mixed with anti-mouse IgG coated magnetic beads (Dynal) for 20 min at 4-8° C. with gentle shaking followed by elimination of labeled non-B cells on the magnetic bead separator. This procedure is repeated once more. The B cells are resuspended in RPMI-1640 with 10% FBS, and kept on ice until further use.

B. Measurement of Antibody Production by Human B Cells

To study antibody production, B cells are aliquoted at 50-75×10³ cells/well into 96-well plate with or without inhibitor, to which IL-2 (100 U/mL) and PANSORBIN® (Calbiochem) Staphylococcus aureus cells (1:90,000) were added. Part of the media is removed after 24-36 hr, and fresh media (with or without inhibitor) and IL-2 is added. Cultures are incubated at 37° C., in the presence of a CO₂ incubator for additional 7 days. Samples from each condition (in triplicate) are removed, and analyzed for IgG and IgM, as measured by ELISA. Briefly, IMMULON® 4 96-well plates are coated (50 μL/well) with either 150 ng/mL donkey antihuman IgG (H+L) (Jackson ImmunoResearch, West Grove Pa.), or 2 ng/mL donkey antihuman IgG+IgM (H+L) (Jackson ImmunoResearch) in bicarbonate buffer, and left overnight at 4° C. After washing three times with phosphate buffered saline containing 0.1% TWEEN®-80 (PBST) (350 μL/well), and blocking with 3% goat serum in PBST (100 μL/well) for 1 hr at room temperature, samples (100 μL/well) of B cell spent media diluted in PBST are added. For IgG plates the dilution range is 1:500 to 1:10000, and for IgM 1:50 to 1:1000. After 1 hr, plates are exposed to biotin-conjugated antihuman IgG (100 ng/mL) or antihuman IgM (200 ng/mL) (Jackson ImmunoResearch) for 30 min, following by streptavidin-HRP (1:20000) for 30 min, and finally, to TMB solution (1:100) with H₂O₂ (1:10000) for 5 min, with 3× PBST washing between steps. Color development is stopped by H₂SO₄ solution, and plates were read on an ELISA plate reader.

Compounds of the present invention inhibited antibody production.

C. Measurement of B Cell Proliferation in Response to Cell Surface IgM Stimulation

In the above experiment, B cells are stimulated using PANSORBIN®. The effect compounds of the present invention on B cell proliferation response when they are stimulated through their cell surface IgM using anti-IgM antibody also was measured. Murine splenocytes (Balb/c) are plated into 96-well microtiter plates at 2×10⁵ cells per well in 10% FBS/RPMI. Appropriate dilutions of test inhibitor in complete medium are added to the cells and the plates are incubated for 30-60 minutes prior to the addition of stimulus. Following the preincubation with test inhibitor, an F(ab′)₂ preparation of goat antibody specific for the μ-chain of mouse IgM is added to the wells at a final concentration of 25 μg/mL. The plates are incubated at 37° C. for 3 days and 1 μCi of [³H]-thymidine is added to each well for the final four hours of culture. The plates are harvested onto fiber filters, washed, and the incorporation of radiolabel is determined using a beta counter (Matrix 96, Packard Instrument Co., Downers Grove, Ill.) and expressed as counts per minute (CPM).

Compounds of the present invention inhibit anti-IgM-stimulated B cell proliferation in a dose-dependent manner Because compounds of the present invention inhibit B cell proliferation, it is envisioned that these compounds and other PI3Kδ inhibitors could be used to suppress undesirable proliferation of B cells in clinical settings. For example, in B cell malignancy, B cells of various stages of differentiation show unregulated proliferation. Based on the results shown above, one can infer that PI3Kδ selective inhibitors could be used to control, limit, or inhibit growth of such cells.

### Example 5

Characterization of Role of PI3Kδ in T Lymphocyte Function

T cell proliferation in response to costimulation of CD3+CD28 is measured. T cells are purified from healthy human blood by negative selection using antibody coated magnetic beads according to the manufacturer's protocol (Dynal) and resuspended in RPMI. The cells are treated with either DMSO or a serial dilution of a present compound in DMSO and plated at 1×10⁵ cells/well on a 96-well plate precoated with goat antimouse IgG. Mouse monoclonal anti-CD3 and anti-CD28 antibodies then are added to each well at 0.2 ng/mL and 0.2 ng/mL, respectively. The plate is incubated at 37° C. for 24 hr and [³H]-thymidine (1 μCi/well) is added. After another 18-hr incubation, the cells are harvested with an automatic cell harvester, washed, and the incorporated radioactivity was quantified.

Although the present PI3Kδ inhibitor compounds inhibited anti-CD3- and anti-CD28-induced proliferation of T cells, an effect is not as strong as an effect on B cells or on some of the functions of neutrophils.

### Example 6

Characterization of Role of PI3Kδ in Osteoclast Function

To analyze the effect of the present PI3Kδ inhibitor compounds on osteoclasts, mouse bone marrow cells are isolated and differentiated to osteoclasts by treating the cells with Macrophage Colony Stimulating Factor⁻¹ (mCSF⁻¹) and Osteoprotegerin Ligand (OPGL) in serum-containing medium (aMEM with 10% heat-inactivated FBS; Sigma) for 3 days. On day four, when the osteoclasts had developed, the medium is removed and cells are harvested. The osteoclasts are plated on dentine slices at 10⁵ cells/well in growth medium, i.e., αMEM containing 1% serum and 2% BSA with 55 μg/mL OPGL and 10 ng/mL mCSF⁻¹. After 3 hr, the medium is changed to 1% serum and 1% BSA, with or without osteopontin (25 μg/mL) and the PI3K inhibitors (100 nM). The medium is changed every 24 hours with fresh osteopontin and the inhibitors. At 72 hr, the medium is removed, and the dentine surfaces are washed with water to remove cell debris and stained with acid hematoxylin. Excess stain is washed and the pit depths are quantitated using confocal microscopy.

The present PI3-kinase inhibitors had an inhibitory effect on osteoclast function. Both the nonspecific inhibitors LY294002 and wortmannin inhibited osteoclast activity. However, the present PI3Kδ inhibitor compounds had a greater effect, and in some cases almost completely inhibited osteoclast activity.

### Example 7

Characterization of Role of PI3Kδ in Basophil Function

Assessment of the effect of a compound of the invention on basophil function is tested using a conventional histamine release assay, generally in accordance with the method described in Miura et al., J Immunol, 162:4198-206 (1999). Briefly, enriched basophils are preincubated with test compounds at several concentrations from 0.1 nM to 1,000 nM for 10 min at 37° C. Then, polyclonal goat antihuman IgE (0.1 μg/mL) or fMLP is added, and allowed to incubate for an additional 30 min Histamine released into the supernatant is measured using an automated fluorometric technique.

A dose-dependent decrease in histamine release was observed for the present compounds when the basophils are stimulated with anti-IgE. This suppression of histamine release was essentially 100% at 1,000 nM. The present compound did not elicit any effect when the basophils are stimulated with fMLP. For comparison, the nonselective PI3K inhibitor LY294002 is tested at 0.1 nM and 10,000 nM, showing close to 100% inhibition of histamine release at the highest concentration.

This indicates that the present inhibitors of PI3Kδ activity can be used to suppress release of histamine, which is one of the mediators of allergy. Since the activity of various PI 3-kinases are required for protein trafficking, secretion, and exocytosis in many cell types, the above suggests that histamine release by other cells, such as mast cells, also can be disrupted by PI 3-kinase delta-selective inhibitors.

### Chemical Synethesis Examples

Specific nonlimiting examples of compounds of the invention are provided below along with exemplary and general routes for their synthesis. It is understood in the art that protecting groups can be employed where necessary in accordance with general principles of synthetic chemistry. These protecting groups are usually removed in the final steps of the synthesis under basic, acidic, or hydrogenolytic conditions readily apparent to persons skilled in the art. By employing appropriate manipulation and protection of any chemical functionalities, synthesis of compounds of structural formula (I) not specifically set forth herein can be accomplished by methods analogous to the schemes set forth below.

Unless otherwise noted, all starting materials were obtained from commercial suppliers and used without further purification. All reactions and chromatography fractions were analyzed by thin-layer chromatography (TLC) on 250 mm silica gel plates, visualized with ultraviolet (UV) light or iodine (I₂) stain. Products and intermediates were purified by flash chromatography or reverse-phase high performance liquid chromatography.

The following abbreviations are used in the synthetic examples: aq (aqueous), RT (room temperature), H₂O (water), HCl (hydrochloric acid), MeOH (methanol), TFA (trifluoroacetic acid), K₂CO₃ (potassium carbonate), SOCl₂ (thionyl chloride), CH₂Cl₂ (methylene chloride), DMF (dimethylformamide), AcOH (acetic acid), KOAc (potassium acetate), TLC (thin layer chromatography), HPLC (high performance liquid chromatography), HATU (O-(7-azabenzotriazol-1-yl)-N,N,N′,N′-tetramethyluronium hexafluorophosphate), POCl₃ (phosphorus oxychloride), NBS (N-bromosuccinamide), CH₃CN (acetonitrile), DIEA (diisopropylethylamine), and NH₃ (ammonia).

### General Procedures

The compounds of the present invention can be prepared by the following schemes and examples. Additional methods and examples that can readily be adapted by those skilled in the art for making the compounds of the invention are disclosed in WO 2005/113554 and in a U.S. Provisional Application entitled THIENOPYRIMIDINONES FOR TREATMENT OF INFLAMMATORY DISORDERS AND CANCERS, by White, et al., filed on Nov. 13, 2006 under attorney docket no. 61608-3000100; and in U.S. Pat. Nos. 6,518,277; 6,667,300; 6,949,535; and 6,800,620, and in published U.S. Patent Application US 2006/0106038.

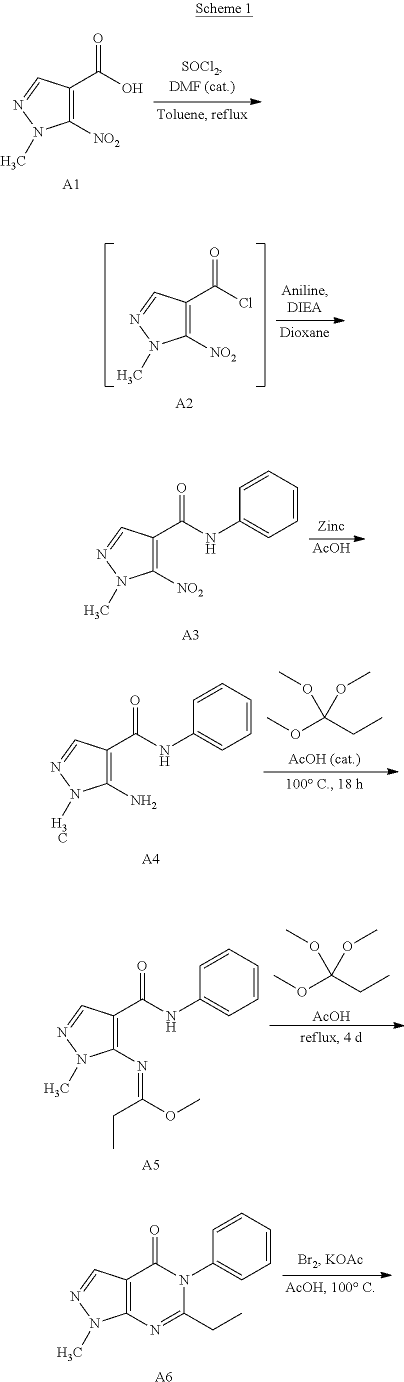

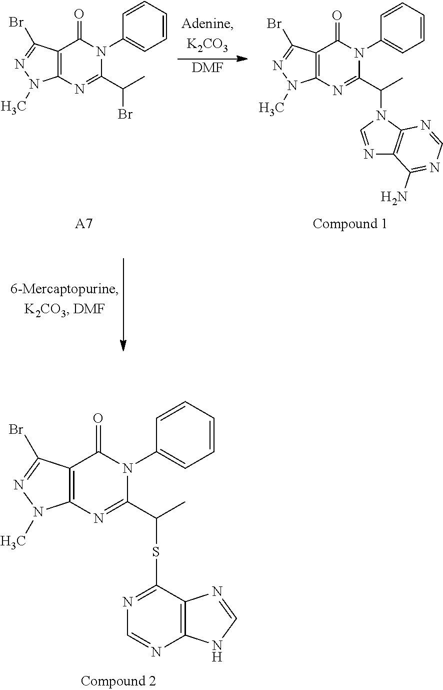

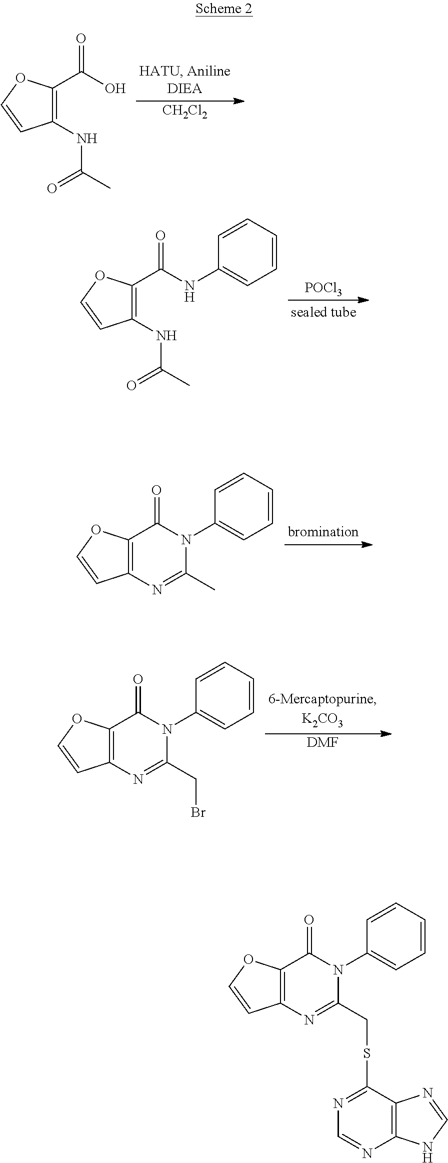

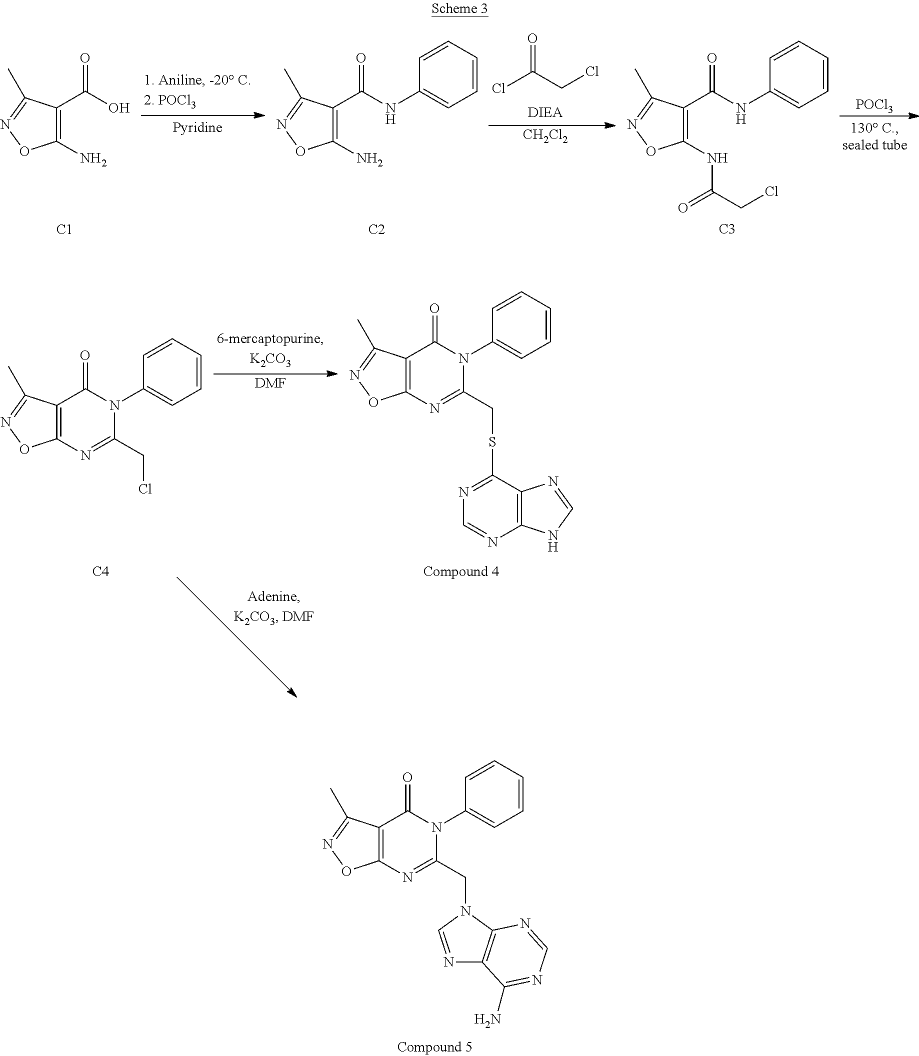

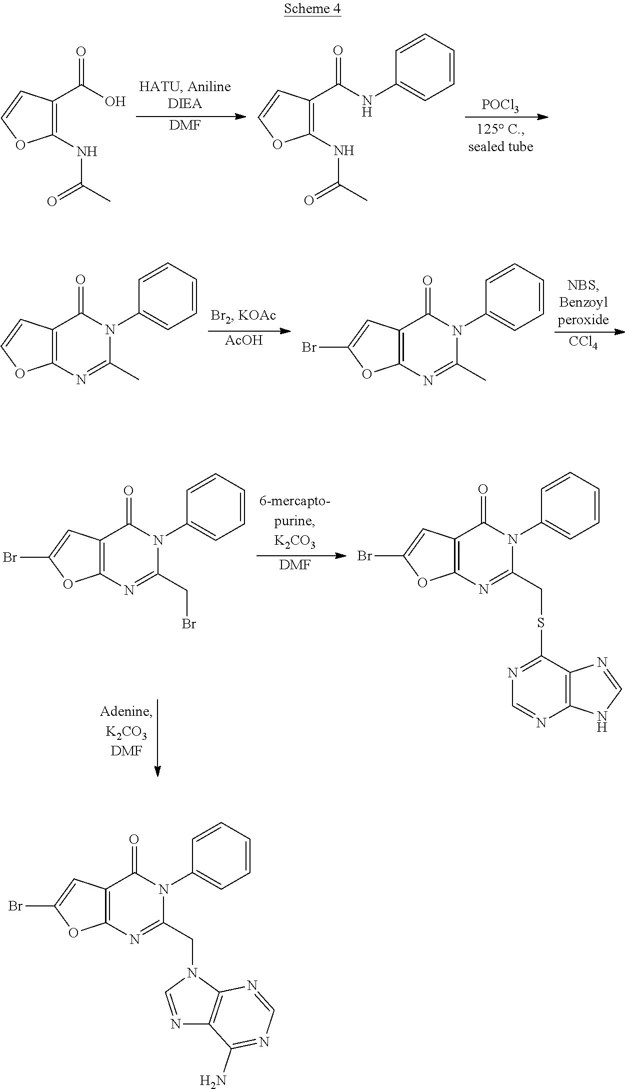

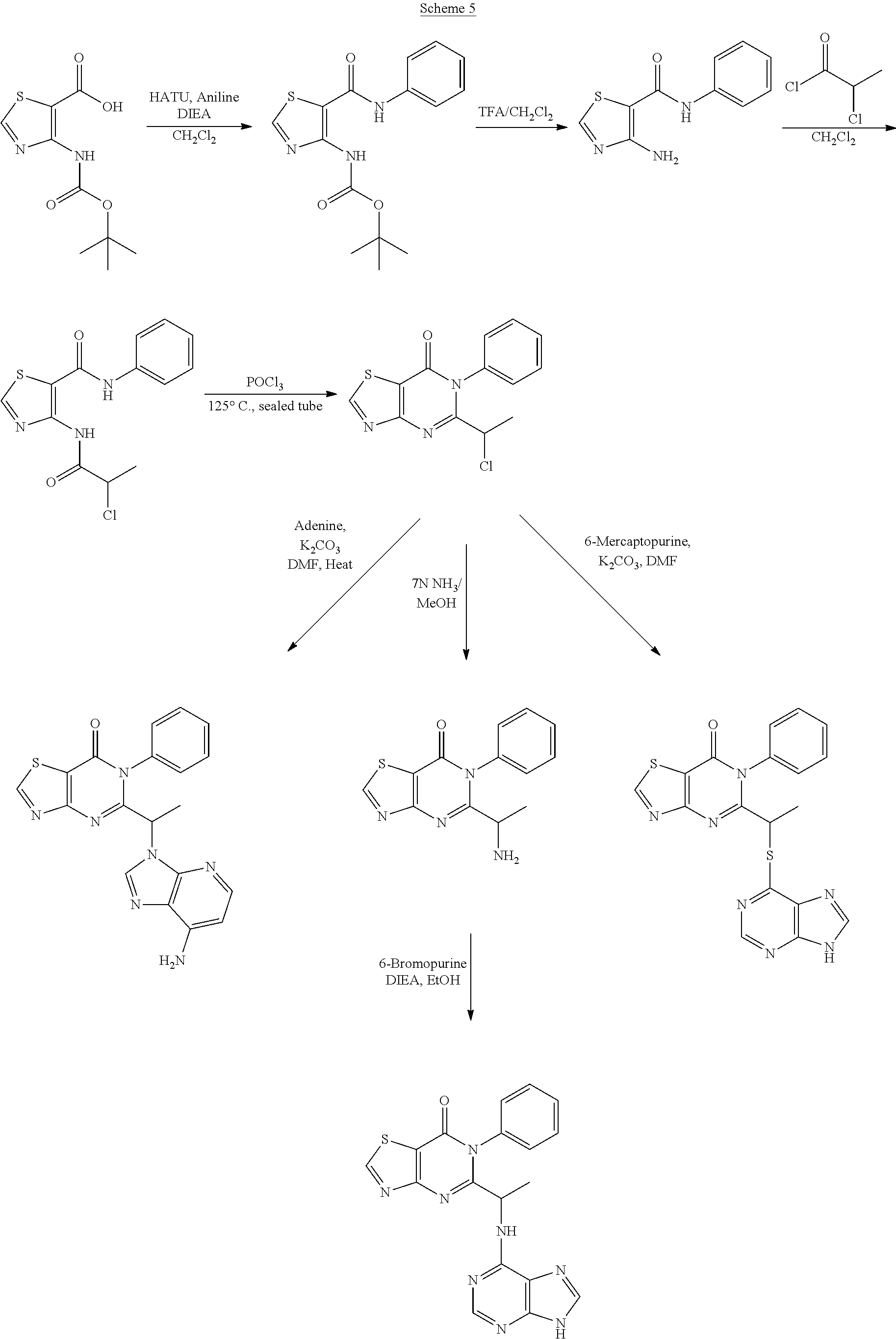

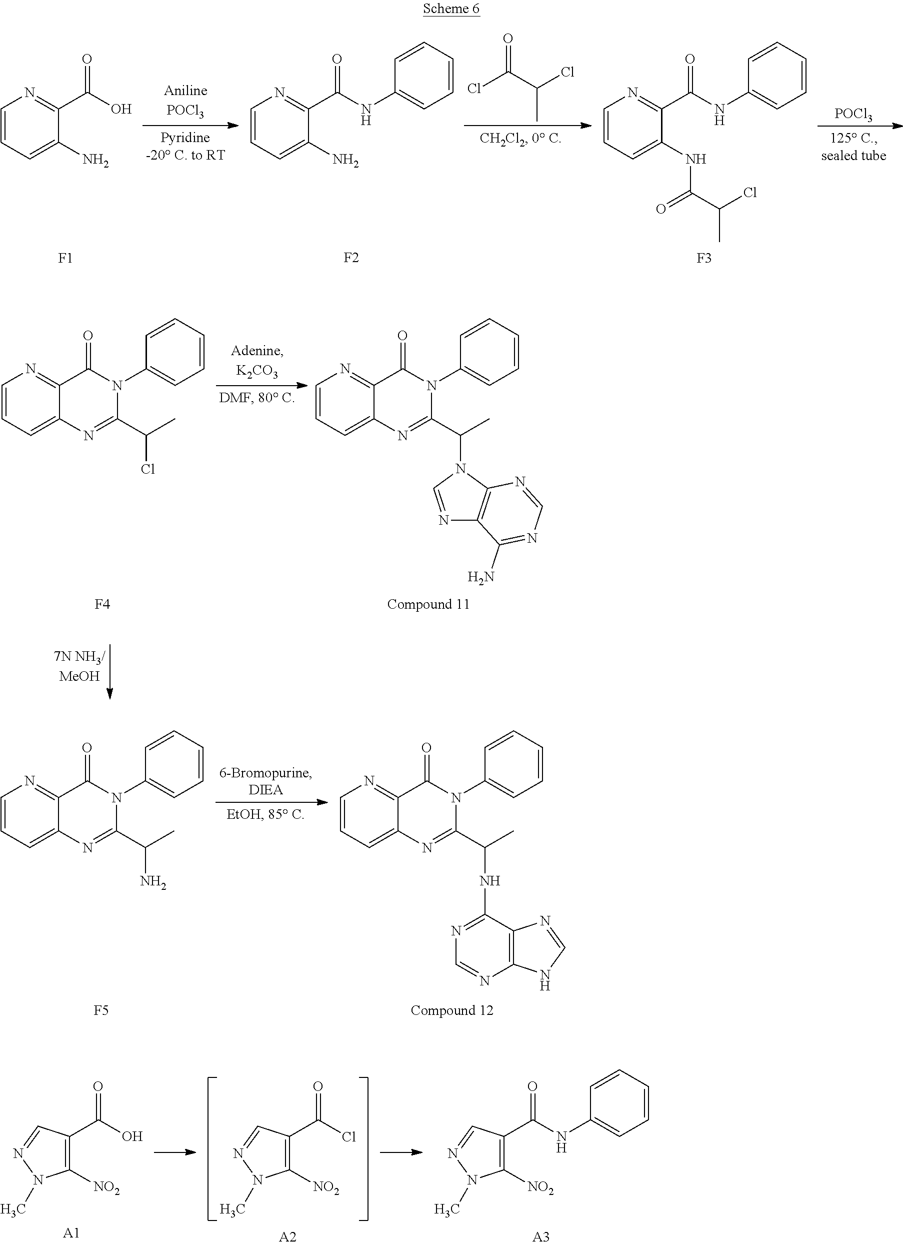

A3: 1-Methyl-5-nitro-1H-pyrazole-4-carboxylic acid phenylamide. A solution of commercially available 1-methyl-5-nitro-1H-pyrazole-4-carboxylic acid (2.5 g, 14.6 mmol) in toluene (10 mL) was treated with DMF (3 drops), followed by addition of thionyl chloride (5.3 mL, 73.0 mmol). The resulting mixture was heated at reflux for 16 h, then cooled to room temperature. The cooled solution then was concentrated by rotary evaporation to provide a residue. The residue was dissolved in dioxane (10 mL), then treated with diisopropyl ethylamine (DIEA) (7.6 mL, 43.8 mmol) followed by the addition of aniline (1.67 mL, 18.3 mmol). The resulting mixture was allowed to stir at room temperature for 16 h, then treated with H₂O (75 mL). A yellow precipitate formed, and was collected by filtering through a fritted glass funnel. The precipitate was dried in a vacuum oven at 50° C. for 2.5 h. Recovered pure product as yellow solid. LC/MS (AP-ESI, AcOH 0.05%) m/z 247 (MH+).

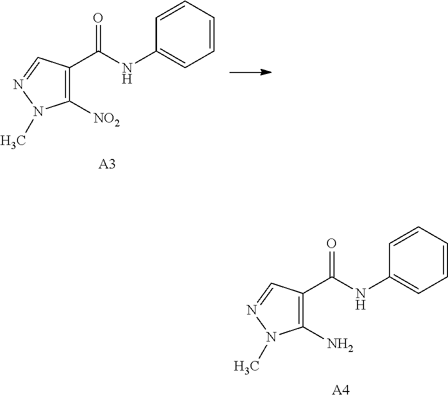

A4: 5-Amino-1-methyl-1H-pyrazole-4-carboxylic acid phenylamide. A solution of 1-methyl-5-nitro-1H-pyrazole-4-carboxylic acid phenylamide (1.5 g, 6.09 mmol) in acetic acid (25 mL) was treated with zinc dust (2.39 g, 36.5 mmol) at room temperature. The resulting mixture was allowed to stir at room temperature for 20 min., then filtered through filter paper. The filtrate was concentrated by rotary evaporation to provide a residue that was dissolved in methylene chloride (20 mL). The solution then was washed with saturated aqueous sodium bicarbonate solution (1×35 mL). Ethyl acetate (75 mL) was added, followed by saturated aqueous sodium chloride solution (20 mL). The organic layer was extracted, and the aqueous layer was extracted with ethyl acetate (3×75 mL). The combined organic extracts were dried over magnesium sulfate, filtered, and concentrated by rotary evaporation to afford the product as a pale-white solid. LC/MS (AP-ESI, AcOH 0.05%) m/z 217 (MH+).

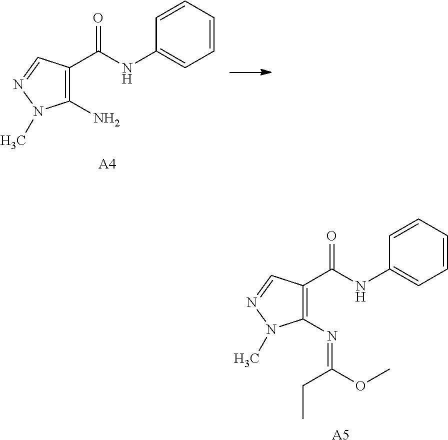

A5: N-(2-Methyl-4-phenylcarbomoyl-2H-pyrazolo-3-yl)-propionimidic acid methyl ester. A solution of 5-amino-1-methyl-1H-pyrazole-4-carboxylic acid phenylamide (1.1 g, 5.09 mmol) in trimethyl orthopropionate was treated with acetic acid (10 drops), and the resulting solution was heated at 100° C. for 18 h. Reaction mixture was concentrated by rotary evaporation, and then dissolved in ethyl acetate (25 mL). The solution then was washed with saturated aqueous sodium bicarbonate solution (1×25 mL), and H₂O (1×25 mL). The organic extract was dried over magnesium sulfate, filtered, and concentrated by rotary evaporation to provide a residue that was treated with hexanes (5 mL). A precipitate formed, and was collected by filtering through filter paper. Recovered pure product as a white solid without further purification. LC/MS (AP-ESI, AcOH 0.05%) m/z 287 (MH+).

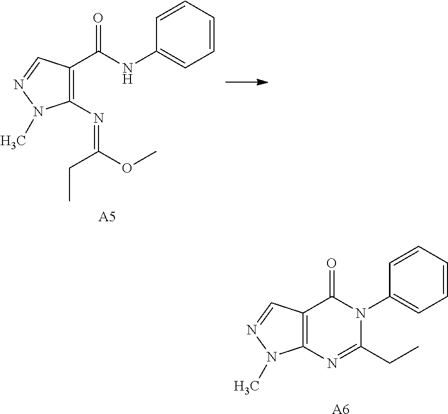

A6: 6-Ethyl-1-methyl-5-phenyl-1,5-dihydro-pyrazolo[3,4-d]pyrimidin-4-one. A solution of N-(2-methyl-4-phenylcarbamoyl-2H-pyrazolo-3-yl)-propionimidic acid methyl ester (760 mgs, 2.65 mmol) in trimethyl orthopropionate (5 mL) was treated with acetic acid (0.30 mL) at room temperature. The resulting solution was heated at reflux for 4 d. Reaction was allowed to cool to room temperature, and a white precipitate formed. The reaction mixture was filtered through filter paper, and the product was recovered as a tan solid. LC/MS (AP-ESI, AcOH 0.05%) m/z 255 (MH+).

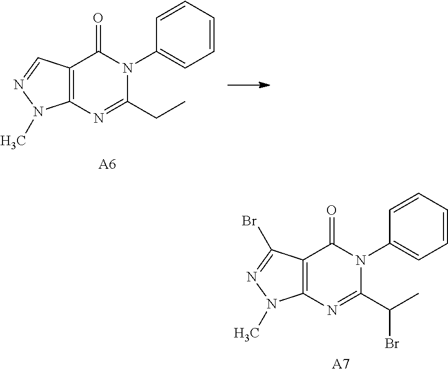

A7: 3-Bromo-6-(1-bromo-ethyl)-1-methyl-5-phenyl-1,5-dihydro-pyrazolo[3,4-d]pyrimidin-4-one. General procedure. A solution of 6-ethyl-1-methyl-5-phenyl-1,5-dihydro-pyrazolo[3,4-d]pyrimidin-4-one in acetic acid (11 mL) was treated with potassium acetate (1.35 g, 13.7 mmol), followed by slow addition of bromine (0.704 mL, 13.7 mL), and the resulting mixture was heated at 100° C. for 7 h. After complete disappearance of starting material by LC/MS, the reaction mixture was poured into a stirring mixture of 1:1 saturated sodium thiosulfate/ethyl acetate (20 mL). The aqueous layer was separated, and the organic layer was washed with H₂O (1×50 mL). The organic extract then was dried over magnesium sulfate, filtered, and concentrated by rotary evaporation to afford crude product (864 mg) as a pale-white solid. Purification by HPLC (C-18 Vydac column 5.0×25 cm, 10-20% CH₃CN/H₂0 containing 0.05% CHCO₂H), and subsequent lyophilizing afforded purified product as a white solid. LC/MS (AP-ESI, AcOH 0.05%) m/z 413 (MH+).

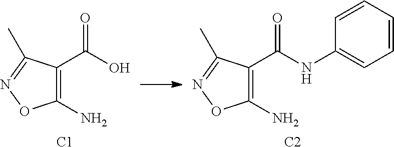

C2: 5-Amino-3-methyl-isoxazole-4-carboxylic acid phenylamide. A solution of commercially available 5-amino-3-methyl-isoxazole-4-carboxylic acid (200 mg, 1.41 mmol) and aniline (321 μL, 3.52 mmol) in pyridine (2.5 mL) was cooled to −20° C., then treated with phosphorous oxychloride (164 μL, 1.76 mmol). After 25 minutes at −20° C., the reaction mixture was allowed to warm to room temperature for 16 h. The reaction mixture was treated with H₂O (30 mL), and the aqueous was extracted with ethyl acetate (2×25 mL). The combined organic extracts were dried over magnesium sulfate, filtered, and concentrated by rotary evaporation to provide the crude product. Purification by HPLC (C-18 Vydac column 5.0×25 cm, 10-20% CH₃CN/H₂0 containing 0.05% AcOH), and subsequent lyophilizing afforded purified product as a white solid. LC/MS (AP-ESI, AcOH 0.05%) m/z 218 (MH+).

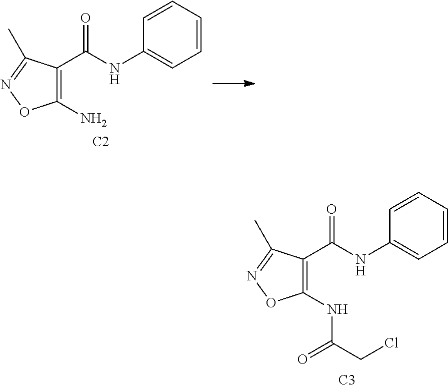

C3: 5-(2-Chloro-acetylamino)-3-methyl-isoxazole-4-carboxylic acid phenylamide. A solution of 5-amino-3-methyl-isoxazole-4-carboxylic acid phenylamide (103 mg, 0.474 mmol) in methylene chloride (2 mL) was treated with chloroacetyl chloride (37 μL, 0.474 mmol), and stirred at room temperature for 16 h. The solution then was treated with DIEA (83 mL, 0.474 mmol). After 20 min., the reaction mixture was treated with an additional equivalent of chloroacetyl chloride (37 μL, 0.474 mmol) and DIEA (83 mL, 0.474 mmol). The solution was allowed to stir at room temperature for 45 min , then treated with 1 N HCl (5 mL). The aqueous phase was extracted with ethyl acetate (1×10 mL). The organic layer was separated, then dried over magnesium sulfate, filtered, and concentrated by rotary evaporation to provide the crude product. Purification by HPLC (C-18 Vydac column 5.0×25 cm, 10-20% CH₃CN/H₂0 containing 0.05% CHCO₂H), and subsequent lyophilizing, provided purified product as a white solid. LC/MS (AP-ESI, AcOH 0.05%) m/z 294 (MH+).

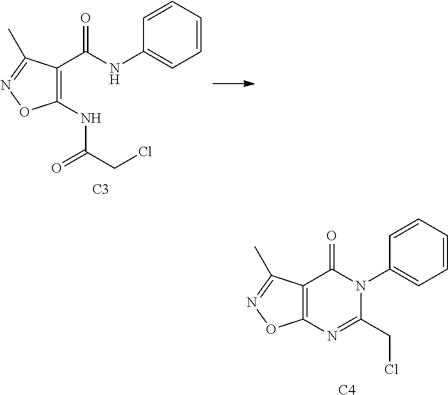

C4: 6-Chloromethyl-3-methyl-5-phenyl-5H-isoxazolo[5,4-d]pyrimidin-4-one. A solution of 5-(2-chloro-acetylamino)-3-methyl-isoxazole-4-carboxylic acid phenylamide (45 mg, 0.153 mmol) in phosphorous oxychloride (7 mL) was heated in a sealed tube at 130° C. for 1 h 40 min. The reaction mixture then was concentrated by rotary evaporation to provide a tan residue. The residue was dissolved in ethyl acetate (10 mL), and treated with saturated aqueous sodium bicarbonate solution (10 mL). The organic layer was separated, dried over magnesium sulfate, filtered, and concentrated by rotary evaporation to provide pure product without purification. LC/MS (AP-ESI, AcOH 0.05%) m/z 276 (MH+).

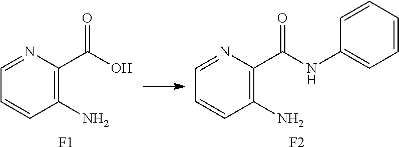

F2: 3-Amino-pyridine-2-carboxylic acid phenylamide. To a stirring mixture of 3-amino picolinic acid (2.0 g, 14.5 mmol) in pyridine (55 mL) was added DIEA (10 drops). Note: starting material was not very soluble. Attempts to increase solubility by heating and sonication improved the solubility marginally. Aniline (3.3 mL. 36.2 mmol) was added, and the reaction mixture was cooled to −20° C. After 10 minutes, POCl₃ and the reaction was allowed to stir for 4 h. Water (50 mL) was added, and the reaction mixture was allowed to stir for 18 h. The reaction mixture then was filtered through a centered glass funnel, and the filtrate was extracted with dichloromethane (2×150 mL). The combined organic extracts were washed with a solution of saturated aqueous sodium bicarbonate (2×200 mL) and H₂O (1×200 mL). The organic extract then was dried over magnesium sulfate, filtered, and concentrated to provide the crude product. Purification on an ISCO automated system eluting with 1-2% ethyl acetate in dichloromethane at 30 mL/min over 1 h provided the pure product. LC/MS (AP-ESI, AcOH 0.05%) m/z 214 (MH+).

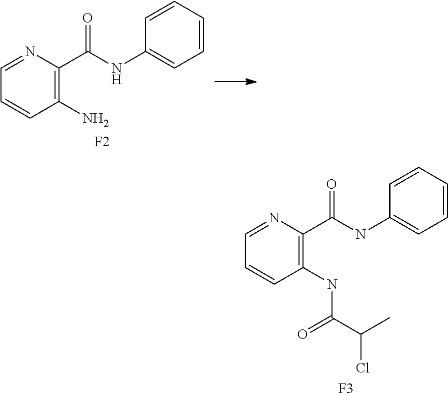

F3: 3-(2-Chloro-propionylamino)-pyridine-2-carboxylic acid phenylamide. To a solution of 3-amino-pyridine-2-carboxylic acid phenylamide (457 mg, 2.14 mmol) in dichloromethane at 0° C. was added 2-chloropropionyl chloride (0.212 mL, 2.14 mmol). After 40 min., the reaction mixture was treated with H₂O (10 mL). The aqueous layer was separated and extracted with dichloromethane (1×15 mL). The organic layers were combined, and dried over magnesium sulfate, filtered, and concentrated to provide the product as a light grey solid without further purification. LC/MS (AP-ESI, AcOH 0.05%) m/z 304 (MH+).

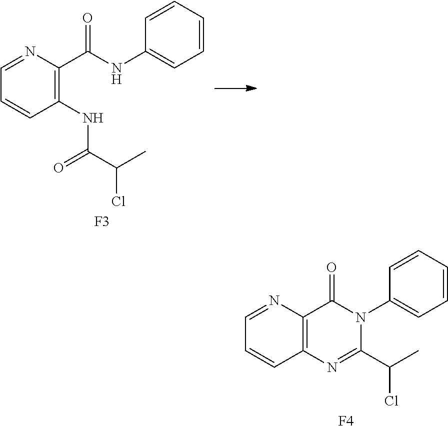

F4: 2-(1-Chloro-ethyl)-3-phenyl-3H-pyrido[3,2-d]pyrimidin-4-one. A solution of 3-(2-chloro-propionylamino)-pyridine-2-carboxylic acid phenylamide (625 mg, 2.06 mmol) in phosphorous oxychloride (5 mL) was heated in a sealed tube at 125° C. for 48 h, then cooled to room temperature and concentrated to provide a residue. The residue was dissolved in ethyl acetate/dichoromethane (2:1, 30 mL), then washed with a solution of saturated sodium bicarbonate (1×50 mL), followed by H₂O (1×50 mL). The organic layer then was dried over magnesium sulfate, filtered, and concentrated to provide the crude product. The crude material was purified on an ISCO automated system eluting with 5-10% methanol in dichloromethane at 30 mL/min over 1 h to provide a pure product. LC/MS (AP-ESI, AcOH 0.05%) m/z 286 (MH+).

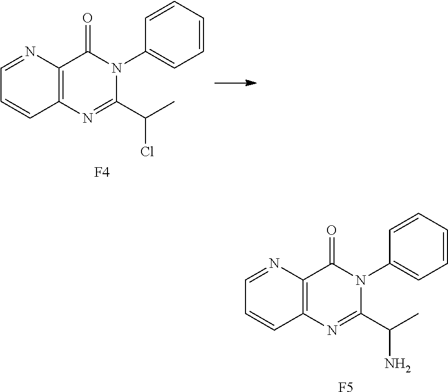

F5: 2-(1-Amino-ethyl)-3-phenyl-3H-pyrido[3,2-d]pyrimidin-4-one. A solution of 2-(1-chloro-ethyl)-3-phenyl-3H-pyrido[3,2-d]pyrimidin-4-one (304 mg, 1.06 mmol) in 7N NH₃/MeOH was heated in a sealed tube at 85° C. for 26.5 h, then cooled to room temperature and concentrated to provide the product. LC/MS (AP-ESI, AcOH 0.05%) m/z 267 (MH+).

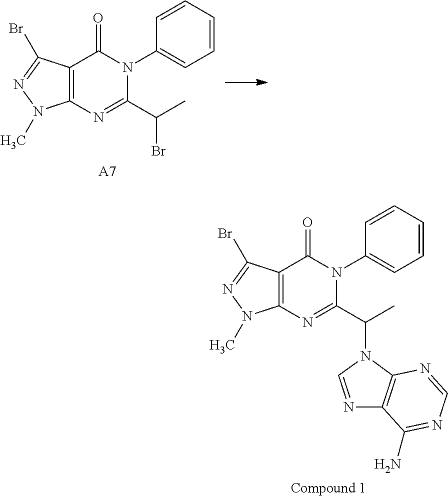

Compound 1: 6-[1-(6-Amino-purin-9-yl)-ethyl]-3-bromo-1-methyl-5-phenyl-1,5-dihydro-pyrazolo[3,4-d]pyrimidin-4-one. General procedure. A stirring solution of 3-bromo-6-(1-bromo-ethyl)-1-methyl-5-phenyl-1,5-dihydro-pyrazolo[3,4-d]pyrimidin-4-one (45 mg, 0.109 mmol) in DMF (2 mL) was treated with adenine (15 mg, 0.111 mmol), followed by the addition of potassium carbonate (15 mg, 0.109 mmol). The resulting mixture was stirred at room temperature for 16 h, then quenched by adding saturated, aqueous sodium chloride solution (5 mL), which gave a white precipitate. After filtering, the product was obtained as a white solid: ¹H NMR (400 MHz, DMSO-d₆) δ 8.26 (apparent fine d, J=1.8 Hz, 1H), 7.97 (apparent fine d, J=2.2 Hz, 1H), 7.59 (m, 2H), 7.44 (t, J=6.6 Hz, 1H), 7.33 (t, J=7.9 Hz, 1H), 7.20 (br s, 2H), 7.07 (d, J=7.3, 1H), 5.43 (q, J=6.6 Hz, 1H), 3.84 (apparent fine d, J=1.8, 3H), 1.70 (d, J=7.0, 3H); LC/MS (AP-ESI, AcOH 0.05%) m/z 468 (MH+).

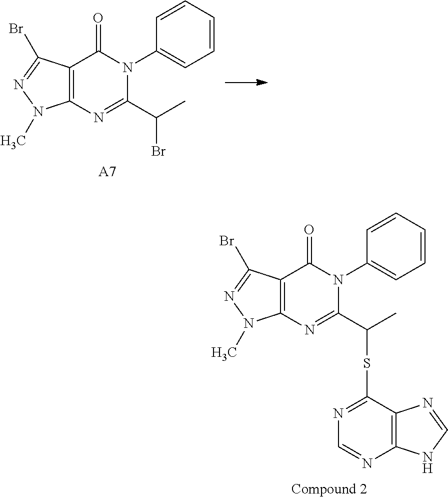

Compound 2: 3-Bromo-1-methyl-5-phenyl-6-[1-(9H-purin-6-ylsulfanyl)-ethyl]-1,5-dihydro-pyrazolo[3,4-d]pyrimidin-4-one. Following the general procedure described for Compound 1, a stirred solution of 3-bromo-6-(1-bromo-ethyl)-1-methyl-5-phenyl-1,5-dihydro-pyrazolo[3,4-d]pyrimidin-4-one (25 mg, 0.06 mmol) in DMF (2 mL) was treated with 6-mercaptopurine monohydrate (11 mg, 0.06 mmol) followed by the addition of potassium carbonate (9 mg, 0.06 mmol). Product was obtained as a yellow solid: ¹H NMR (400 MHz, DMSO-d₆) δ 8.42 (s, 1H), 8.38 (fine d, J=2.6 Hz, 1H), 7.54 (br s, 2H), 7.32 (m, 1H), 7.25 (d, J=8.1, 1H), 7.08 (t, J=7.7, 1H), 5.08 (q, J=7.2, 1H), 3.92 (apparent fine d, J=2.8 Hz, 1H), 1.68 (d, J=6.8, 3H); LC/MS (AP-ESI, AcOH 0.05%) m/z 485 (MH+).

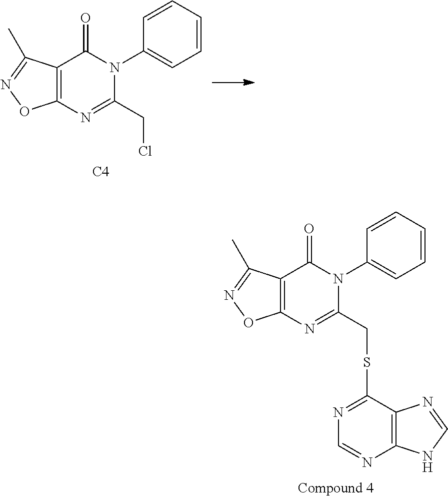

Compound 4: 3-Methyl-5-phenyl-6-(9H-purin-6-ylsulfanylmethyl)-5H-isoxazolo[5,4-d]pyrimidin-4-one. Following the general procedure described for Compound 1, a stirred solution of 6-chloromethyl-3-methyl-5-phenyl-5H-isoxazolo[5,4-d]pyrimidin-4-one (20 mg, 0.073 mmol) in DMF (500 μL) was treated with 6-mercaptopurine monohydrate (12.5 mg, 0.073 mmol), followed by the addition of potassium carbonate (10 mg, 0.073 mmol). The crude reaction mixture was purified via HPLC (C-18 Luna column 1×18 mm, 10-20% CH₃CN/H₂O containing 0.05% CHCO₂H). The product was obtained as a fluffy white solid after lyophilizing: ¹H NMR (400 MHz, DMSO-d₆) δ 8.55 (s, 1H), 8.45 (br s, 1H), 7.55 (m, 5H), 4.40 (s, 2H), 2.44 (s, 3H); LC/MS (AP-ESI, AcOH 0.05%) m/z (MH+) 392.

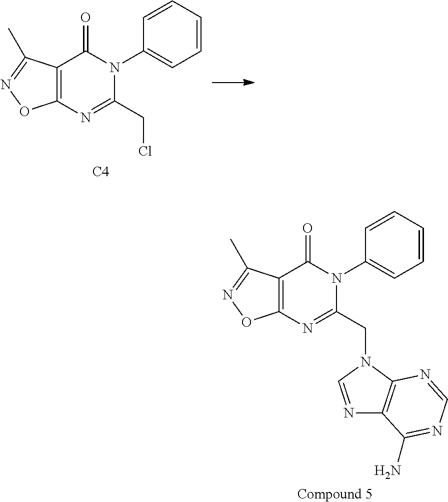

Compound 5: 6-(6-Amino-purin-9-ylmethyl)-3-methyl-5-phenyl-5H-is oxazolo[5,4-d]pyrimidin-4-one. Following the general procedure described for Compound 1, a stirred solution of 6-chloromethyl-3-methyl-5-phenyl-5H-isoxazolo[5,4-d]pyrimidin-4-one (20 mg, 0.073 mmol) in DMF (500 μL) was treated with adenine (10 mg, 0.111 mmol) followed by the addition of potassium carbonate (15 mg, 0.109 mmol). The crude reaction mixture was purified via HPLC (C-18 Luna column 1×18 mm, 10-20% CH₃CN/H₂0 containing 0.05% CHCO₂H). The product was obtained as a fluffy white solid after lyophilizing: ¹H NMR (400 MHz, DMSO-d₆) δ 8.25 (s, 1H), 8.20 (s, 1H), 7.63 (m, 5H), 5.14 (s, 2H), 2.47 (s, 3H); LC/MS (AP-ESI, AcOH 0.05%) m/z (MH+) 375.

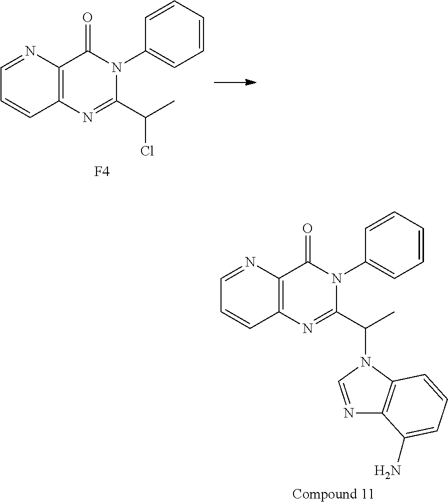

Compound 11: 2-[1-(4-Amino-benzoimidazol-1-yl)-ethyl]-3-phenyl-3H-pyrido[3,2-d]pyrimidin-4-one. To a stirring solution of 2-(1-chloro-ethyl)-3-phenyl-3H-pyrido[3,2-d]pyrimidin-4-one (94 mg, 0.329 mmol) in DMF (500 μL) was added adenine (66 mg, 0.494 mmol), followed by the addition of potassium carbonate (45 mg, 0.329 mmol). The resulting mixture then was heated at 80° C. for 1 h, cooled to room temperature and treated with H₂O (10 mL). The resulting mixture was concentrated to provide the crude product, which then was purified by HPLC (C-18 Luna column 250×21.20 mm, 10-20% CH₃CN/H₂O containing 0.05% CF₃CO₂H). The product was obtained as a white solid after lyophilizing: ¹H NMR (400 MHz, DMSO-d₆) δ 8.83 (m, 1H), 8.45 (fine d, J=1.2 Hz, 1H), 8.20 (fine d, J=1.6 Hz, 1H), 8.10 (m, 1H), 7.85 (m, 1H), 7.70 (d, J=8.2 Hz, 1H), 7.59 (t, J=7.8 Hz, 1H), 7.44 (t, J=7.4 Hz, 1H), 7.34 (t, J=7.4 Hz, 1H), 7.12 (d, J=8.2 Hz, 1H), 5.51 (q, J=7.0 Hz, 1H), 1.77 (d, J=6.8 Hz, 3H). LC/MS (AP-ESI, AcOH 0.05%) m/z 385 (MH+).

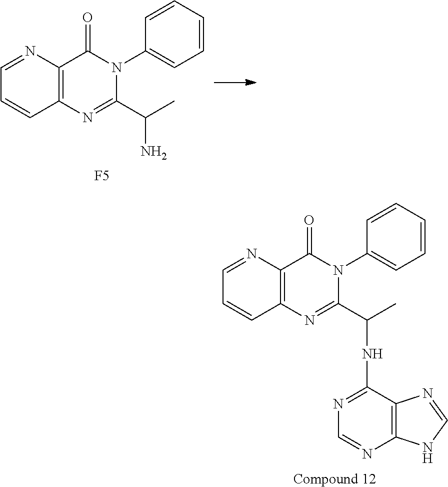

Compound 12: 3-Phenyl-2-[1-(9H-purin-6-ylamino)-ethyl]-3H-pyrido[3,2-d]pyrimidin-4-one. To a solution of 2-(1-amino-ethyl)-3-phenyl-3H-pyrido[3,2-d]pyrimidin-4-one (328 mg, 1.23 mmol) in ethanol (1 mL) was added 6-bromopurine (245 mg, 1.23 mmol) and DIEA (429 μL, 2.46 mmol). The resulting solution was heated at 85° C. for 24 h, then cooled to room temperature and concentrated to provide the crude product. The crude material then was purified by HPLC (C-18 Luna column 250×21.20 mm, 10-20% CH₃CN/H₂O containing 0.05% CF₃CO₂H). The product was obtained after lyophilizing LC/MS (AP-ESI, AcOH 0.05%) m/z 385 (MH+).

### Example 9

Biochemical Assays of PI3K Potency, Selectivity, and Bioavailability

Biochemical Assay Using 20 μM ATP

Using the method described in Example 2, above, compounds of the invention were tested for inhibitory activity and potency against PI3Kδ, and for selectivity for PI3Kδ versus other Class I PI3K isozymes. IC₅₀ values (μM) are given for PI3Kα (“Alpha”), PI3Kβ (“Beta”), PI3γ (“Gamma”), and PI3Kδ (“Delta”). To illustrate selectivity of the compounds, the ratios of the IC₅₀ values of the compounds for PI3Kα, PI3Kβ, and PI3Kδ relative to PI3Kδ are given, respectively, as “Alpha/Delta Ratio,” “Beta/Delta Ratio,” and “Gamma/Delta Ratio.”

The initial selectivity assays were done identically to the selectivity assay protocol in Example 2, except using 100 μL Ecoscint for radiolabel detection. Subsequent selectivity assays were done similarly using the same 3× substrate stocks except they contained 0.05 mCi/mL γ[³²P]ATP and 3 mM PIP2. Subsequent selectivity assays also used the same 3× enzyme stocks, except they now contained 3 nM of any given PI3K isoform.

For all selectivity assays, the test compounds were weighed out and dissolved into 10-50 mM stocks in 100% DMSO (depending on their respective solubilities) and stored at −20° C. Compounds were thawed (to room temperature or 37° C.), diluted to 300 μM in water from which a 3-fold dilution series into water was done. From these dilutions, 20 μL was added into the assay wells alongside water blanks used for the enzyme (positive) control and the no enzyme (background) control. The rest of the assay was essentially done according to the selectivity assay protocol in Example 2.

### Example 10

Cell-Based Assay Data for Inhibitors of PI3Kδ Activity

Using the methods described in Example 3, above, compounds of the invention were tested for inhibitory activity and potency in an assay of neutrophil (PMN) elastase release.

Compounds of the invention were tested and shown to be selective inhibitors of PI3Kδ; some of the specific compounds within the scope of the invention are shown in Table 1, along with in vitro activity data for selected compounds.

TABLE 1
Selected Compounds and Activity Data.

| Compound | | In Vitro I-50 |
|---|---|---|
| No. | Structure | (micromolar) |
| 1 | 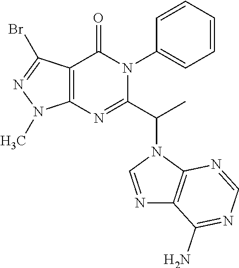 | 0.665 |
| 2 | 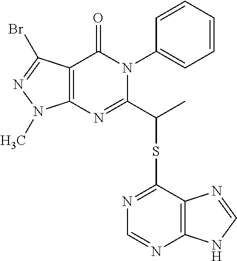 | 6.0 |
| 3 | 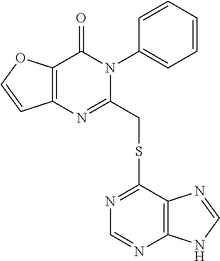 | |
| 4 | 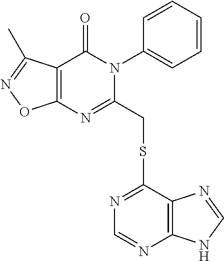 | 1.2 |
| 5 | 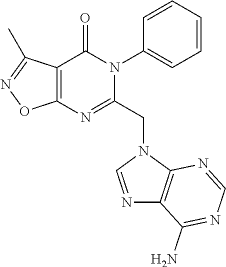 | 6.4 |
| 6 | 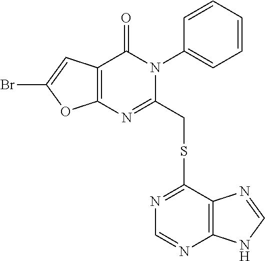 | |
| 7 | 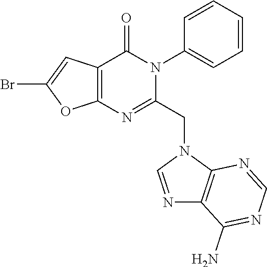 | |
| 8 | 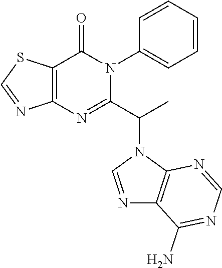 | |
| 9 | 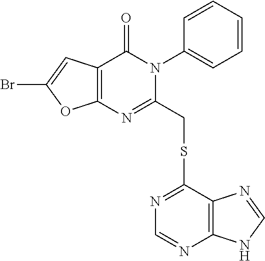 | |
| 10 | 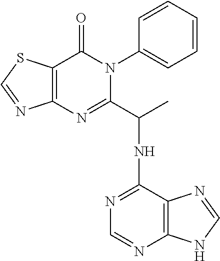 | |
| 11 | 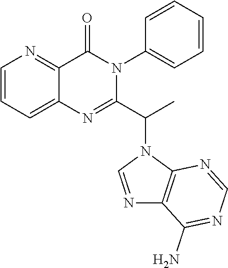 | 0.665 |
| 12 | 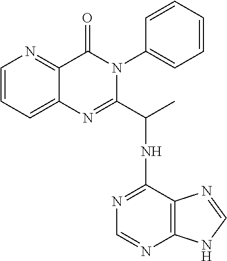 | |

While the present invention has been described with specific reference to certain embodiments for purposes of clarity and understanding, it will be apparent to the skilled artisan that further changes and modifications can be practiced within the scope of the invention as it is defined in the claims set forth below. Accordingly, no limitations should be placed on the invention other than those specifically recited in the claims.

1. A compound of formula (I): 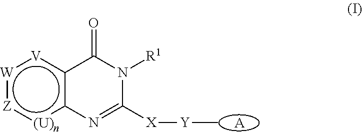
wherein U, V, W, and Z, independently, are selected from the group consisting of CR^{a}, N, NR^{b}, and O,
or wherein at least one of U, V, W and Z is N, and the others of U, V, W and Z are selected from the group consisting of CR^{a}, NR^{b}, S, and O,
and wherein at least one, but not all, of U, V, W, and Z is different from CR^{a};
A is an optionally substituted monocyclic or bicyclic ring system containing at least two nitrogen atoms as ring members, and at least one ring of the system is aromatic;
X is selected from the group consisting of C(R^{c})₂, C(R^{c})₂C(R^{c})₂, CH₂CHR^{c}, CHR^{c}CHR^{c}, CHR^{c}CH₂, CH═C(R^{c}), C(R^{c})═C(R^{c}) and C(R^{c})═CH;
Y is selected from the group consisting of null (i.e., a bond), S, SO, SO₂, NH, N(R^{c}), O, C(═O), OC(═O), C(═O)O, and NHC(═O)CH₂S;
R¹ is selected from the group consisting of H, substituted or unsubstituted C₁₋₁₀alkyl, substituted or unsubstituted C₂₋₁₀alkenyl, substituted or unsubstituted C₂₋₁₀alkynyl, substituted or unsubstituted C₁₋₆perfluoroalkyl, substituted or unsubstituted C₃₋₈cycloalkyl, substituted or unsubstituted C₃₋₈heterocycloalkyl, substituted or unsubstituted C₁₋₄alkyleneC₃₋₈cycloalkyl, substituted or unsubstituted aryl, substituted or unsubstituted heteroaryl, substituted or unsubstituted arylC₁₋₄alkyleneOR^{e}, substituted or unsubstituted heteroarylC₁₋₄alkyleneN(R^{d})₂, substituted or unsubstituted heteroarylC₁₋₄alkyleneOR^{e}, substituted or unsubstituted C₁₋₃alkyleneheteroaryl, substituted or unsubstituted C₁₋₃alkylenearyl, substituted or unsubstituted arylC₁₋₆alkyl, arylC₁₋₄alkyleneN(R^{d})₂, C₁₋₄alkyleneC(═O)C₁₋₄alkylenearyl, C₁₋₄alkyleneC(═O)C₁₋₄alkyleneheteroaryl, C₁₋₄alkyleneC(═O)heteroaryl, C₁₋₄alkyleneC(═O)N(R^{d})₂, C₁₋₆alkyleneOR^{d}, C₁₋₄alkyleneNR^{a}C(═O)R^{d}, C₁₋₄alkyleneOC₁₋₄alkyleneOR^{d}, C₁₋₄alkyleneN(R^{d})₂, C₁₋₄alkyleneC(═O)OR^{d}, and C₁₋₄alkyleneOC₁₋₄alkyleneC(═O)OR^{d};
R^{a}, independently, is selected from the group consisting of H, substituted or unsubstituted C₁₋₆alkyl, substituted or unsubstituted C₃₋₈cycloalkyl, substituted or unsubstituted C₃₋₈heterocycloalkyl, substituted or unsubstituted aryl, C₁₋₃alkylenearyl, substituted or unsubstituted heteroaryl, substituted or unsubstituted heteroarylC₁₋₃alkyl, substituted or unsubstituted C₁₋₃alkyleneheteroaryl, halo, NHC(═O)C₁₋₃alkyleneN(R^{d})₂, NO₂, OR^{e}, CF₃, OCF₃, N(R^{d})₂, CN, OC(═O)R^{d}, C(═O)R^{d}, C(═O)OR^{d}, arylOR^{e}, NR^{d}C(═O)C₁₋₃alkyleneC(═O)OR^{d}, arylOC₁₋₃alkyleneN(R^{d})₂, arylOC(═O)R^{d}, C₁₋₄alkyleneC(═O)OR^{d}, OC₁₋₄alkyleneC(═O)OR^{d}, C₁₋₄alkyleneOC₁₋₄alkyleneC(═O)OR^{d}, C(═O)NR^{d}SO₂R^{d}, C₁₋₄alkyleneN(R^{d})₂, C₂₋₆alkenyleneN(R^{d})₂, C(═O)NR^{d}C₁₋₄alkyleneOR^{e}, C(═O)NR^{d}C₁₋₄alkyleneheteroaryl, OC₁₋₄alkyleneN(R^{d})₂, OC₁₋₄alkyleneCH(OR^{e})CH₂N(R^{d})₂, OC₁₋₄alkyleneheteroaryl, OC₂₋₄alkyleneOR^{e}, OC₂₋₄alkyleneNR^{d}C(═O)OR^{d}, NR^{a}C₁₋₄alkyleneN(R^{d})₂, NR^{a}C═O)R^{d} NR^{a}(C═O)R^{d}, NR^{a}C(═O)N(R^{d})₂, N(SO₂C₁₋₄alkyl)₂, NR^{a}(SO₂C₁₋₄alkyl), SO₂N(R^{d})₂, OSO₂CF₃, C₁₋₃alkylenearyl, C₁₋₄alkyleneheteroaryl, C₁₋₆alkyleneOR^{e}, C(═O)N(R^{d})₂, NHC(═O)C₁₋₃alkylenearyl, arylOC₁₋₃alkyleneN(R^{d})₂, arylOC(═O)R^{d}, NHC(═O)C₁₋₃alkyleneC₃₋₈heterocycloalkyl, NHC(═O)C₁₋₃alkyleneheteroaryl, OC₁₋₄alkyleneOC₁₋₃alkyleneC(═O)OR^{d}, C(═O)C₁₋₄alkyleneheteroaryl, and NHC(═O)haloC₁₋₆alkyl;
R^{b} is selected from the group consisting of null, H, substituted or unsubstituted C₁₋₆alkyl, substituted or unsubstituted C₃₋₈cycloalkyl, substituted or unsubstituted C₃₋₈heterocycloalkyl, substituted or unsubstituted aryl, substituted or unsubstituted arylC₁₋₃alkyl, C₁₋₃alkylenearyl, substituted or unsubstituted heteroaryl, heteroarylC₁₋₃alkyl, substituted or unsubstituted C₁₋₃alkyleneheteroaryl, C(═O)R^{d}, C(═O)OR^{d}, arylOR^{e}, arylOC₁₋₃alkyleneN(R^{d})₂, arylOC(═O)R^{d}, C₁₋₄alkyleneC(═O)OR^{d}, C₁₋₄alkyleneOC₁₋₄alkyleneC(═O)OR^{d}, C(═O)NR^{d}SO₂R^{d}, C₁₋₄alkyleneN(R^{d})₂, C₂₋₆alkenyleneN(R^{d})₂, C(═O)NR^{d}C₁₋₄alkyleneOR^{c}, C(═O)NR_{d}C₁₋₄alkyleneheteroaryl, SO₂N(R^{d})₂, C₁₋₃alkylenearyl, C₁₋₄alkyleneheteroaryl, C₁₋₆alkyleneOR^{e}, C₁₋₃alkyleneN(R^{d})₂, C(═O)N(R^{d})₂, arylOC₁₋₃alkyleneN(R^{d})₂, arylOC(═O)R^{d}, and C(═O)C₁₋₄alkyleneheteroaryl;
R^{c}, independently, is selected from the group consisting of H, substituted or unsubstituted C₁₋₁₀alkyl, substituted or unsubstituted C₃₋₈cycloalkyl, substituted or unsubstituted C₃₋₈heterocycloalkyl, substituted or unsubstituted C₁₋₄alkyleneN(R^{d})₂, substituted or unsubstituted C₁₋₃alkyleneheteroC₁₋₃alkyl, substituted or unsubstituted arylheteroC₁₋₃alkyl, substituted or unsubstituted aryl, substituted or unsubstituted heteroaryl, substituted or unsubstituted arylC₁₋₃alkyl, substituted or unsubstituted heteroarylC₁₋₃alkyl, C₁₋₃alkylenearyl, substituted or unsubstituted C₁₋₃alkyleneheteroaryl, C(═O)R^{d}, and C(═O)OR^{d},
or two R^{c} on the same atom or on adjacent connected atoms can cyclize to form a ring having 3-8 ring members, which ring is optionally substituted and may include up to two heteroatoms selected from NR^{d}, O and S as ring members;
R^{d} is selected from the group consisting of H, substituted or unsubstituted C₁₋₁₀alkyl, substituted or unsubstituted C₂₋₁₀alkenyl, substituted or unsubstituted C₂₋₁₀alkynyl, substituted or unsubstituted C₃₋₈cycloalkyl, substituted or unsubstituted C₃₋₈heterocycloalkyl, substituted or unsubstituted C₁₋₃alkyleneN(R^{e})₂ aryl, substituted or unsubstituted arylC₁₋₃alkyl, substituted or unsubstituted C₁₋₃alkylenearyl, substituted or unsubstituted heteroaryl, substituted or unsubstituted heteroarylC₁₋₃alkyl, and substituted or unsubstituted C₁₋₃alkyleneheteroaryl;
or two R^{d} groups are taken together with the nitrogen to which they are attached to form a 5- or 6-membered ring, optionally containing a second heteroatom that is N, O or S;
R^{e} is selected from the group consisting of H, substituted or unsubstituted C₁₋₆alkyl, substituted or unsubstituted C₃₋₈cycloalkyl, substituted or unsubstituted aryl, and substituted or unsubstituted heteroaryl,
or two R^{e} groups are taken together with the nitrogen to which they are attached to form a 5- or 6-membered ring, optionally containing a second heteroatom that is N, O or S;
said A, R¹, R^{a}, R^{b}, R^{c}, and R^{d}, independently, are optionally substituted with one to three substituents selected from the group consisting of C₁₋₁₀alkyl, C₂₋₁₀alkenyl, C₂₋₁₀alkynyl, C₃₋₈cycloalkyl, C₃₋₈heterocycloalkyl, C₃₋₆alkyleneOR^{c}, C₁₋₄alkyleneN(R^{c})₂, aryl, C₁₋₃alkylenearyl, heteroaryl, C(═O)OR^{e}, C(═O)R^{e}, OC(═O)R^{e}, halo, CN, CF₃, NO₂, N(R^{e})₂, OR^{e}, OC₁₋₆perfluoroalkyl, OC(═O)N(R^{e})₂, C(═O)N(R^{e})₂, SR^{e}, SO₂R^{e}, SO₃R^{e}, oxo(═O), and CHO; and
n is 0 or 1; or
a pharmaceutically acceptable salt thereof.

2. The compound of claim 1 , wherein n is 0 and one of V, W and Z is NR^{b}.

3. The compound of in claim 1, wherein n is 0 and one of V, W and Z is O.

4. The compound of claim 1, wherein n is 0 and one of V, W and Z is S.

5. The compound of claim 1, wherein n is 1 and one of V, W, U and Z is N and the others are CR^{a}.

6. The compound of formula (I), wherein n is 1 and two of V, W, U and Z are N and the others are CR^{a}.

7. The compound of claim 1, wherein A is an optionally substituted bicyclic aromatic group.

8. The compound of claim 7, wherein A comprises a pyrimidine ring or a pyrimidinone ring, and wherein A is optionally substituted by up to three substituents.

9. The compound of claim 8, wherein R¹ is an optionally substituted ring selected from the group consisting of phenyl, heteroaryl and C₃₋₈ cycloalkyl.

10. The compound of claim 9, wherein X is C(R^{c})₂.

11. The compound of claim 9, wherein Y is a bond, NH or S.

12. The compound of claim 10, wherein X is CH₂ or C(R^{c})H, wherein R^{c} is C1-C4 alkyl.

13. The compound of claim 12, wherein X is C(R^{c})H and is in the S configuration.

14. The compound of claim 9, wherein X and Y are cyclized together to form a ring of the formula:

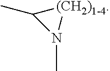

15. The compound of claim 1, wherein A is a purine group that is optionally substituted with up to three substituents selected from halo, NH₂, NHMe, NMe₂, OH, SMe, and Me.

16. The compound of claim 1, wherein X is CHMe or CHEt.

17. The compound of claim 15, wherein R¹ is phenyl that is optionally substituted with one to three substituents selected from the group consisting of halo, OR^{e}, C₁₋₆alkyl, C₂₋₆alkenyl, C₂₋₆alkynyl, aryl, C₃₋₈heterocycloalkyl, heteroaryl, CF₃, NO₂, N(R^{e})₂, C(═O)OR^{e}, SO₂N(R^{a})₂, CN, C(═O)R^{e}, C(═O)N(R^{e})₂, C₁₋₄alkyleneN(R^{e})₂, OC₁₋₄perfluoroalkyl, oxo, and CHO.

18. A pharmaceutical composition comprising a compound of claim 1, admixed with at least one pharmaceutically acceptable excipient.

19. A method of disrupting leukocyte function comprising contacting the leukocytes with an effective amount of a compound of claim 1.

20. A method to treat a subject diagnosed with leukemia, comprising administering to said subject an effective amount of a compound of claim 1.

21. A method to treat a subject diagnosed with lymphoma, comprising administering to said subject an effective amount of a compound of claim 1.

22. A method to treat a subject diagnosed with an immunological disorder, comprising administering to said subject an effective amount of a compound of claim 1.

23. The method of claim 22, wherein the immunological disorder is selected from asthma, rheumatoid arthritis, multiple sclerosis and lupus.

24. A method to treat a subject diagnosed with hypertension, comprising administering to said subject an effective amount of a compound of claim 1.

25. A method to treat a subject diagnosed with a carcinoma or sarcoma, comprising administering to said subject an effective amount of a compound of claim 17.

26. A method to treat a subject diagnosed with a bone resorption disorder, comprising administering to said subject an effective amount of a compound of claim 1.

27. A method of inhibiting kinase activity of a phosphatidylinositol 3-kinase delta polypeptide comprising contacting the polypeptide with a compound of claim 1.
